(19) United States
(12) Patent Application Publication
(10) Pub. No.: US 2025/0200747 A1
(43) Pub. Date: Jun. 19, 2025

(54) IMAGE PROCESSING DEVICE, IMAGE PROCESSING METHOD, AND RECORDING MEDIUM

(71) Applicant: OLYMPUS MEDICAL SYSTEMS CORP., Tokyo (JP)

(72) Inventors: Kenro OSAWA, Tokyo (JP); Munenori FUKUNISHI, Tokyo (JP); Naoki FUKATSU, Tokyo (JP)

(73) Assignee: OLYMPUS MEDICAL SYSTEMS CORP., Tokyo (JP)

(21) Appl. No.: 18/980,318

(22) Filed: Dec. 13, 2024

Related U.S. Application Data
(60) Provisional application No. 63/610,604, filed on Dec. 15, 2023.

(51) Int. Cl. G06T 7/00; G06T 5/50; G16H 30/20; G16H 30/40

(52) U.S. Cl. CPC G06T 7/0012; G06T 5/50; G16H 30/20; G16H 30/40; G06T 2207/10068; G06T 2207/20084; G06T 2207/20221; G06T 2207/30004

(57) ABSTRACT

An image processing device includes: one or more processors comprising hardware, wherein the one or more processors are configured to: input a captured image captured by an endoscope in a body cavity of a subject to a Convolutional Neural Network (CNN) using a trained model, the trained model having training data in which each of training images is associated with category information including a lumen direction of a region outside the each of training images in which the lumen is present; estimate, based on the CNN, category information including a lumen direction of a region outside the captured image in which the lumen is likely to be present; and output the category information estimated.

### CROSS-REFERENCE TO RELATED APPLICATION

This application is based on and claims priority under 35 U.S.C. § 119 to U.S. Provisional Application No. 63/610,604, filed Dec. 15, 2023, the entire contents of which are incorporated herein by reference.

### BACKGROUND

The present disclosure relates to an information processing apparatus, an information processing method, and a recording medium.

In the related art, with respect to an endoscope, a technology for searching for a region to be observed and a lumen direction for insertion to reduce a time taken to resume original operation and improve convenience even when an observation imaging object in a subject is missing or an insertion direction is missing has been known (for example, see Japanese Patent No. 6577031). In this technology, coordinates of an observation position that indicates an innermost position of a lumen are identified from a group of chronological images, and when a coordinate position is not identified in an image of a current frame, a corresponding point between an image of a past frame previous to the current frame and the image of the current frame is detected, coordinate transformation is performed to obtain the coordinate position in the image of the current frame from the detection result, and information on a lumen direction is displayed together with the image of the current frame.

### SUMMARY

In some embodiments, an image processing device includes: one or more processors comprising hardware, wherein the one or more processors are configured to: input a captured image captured by an endoscope in a body cavity of a subject to a Convolutional Neural Network (CNN) using a trained model, the trained model having training data in which each of training images is associated with category information including a lumen direction of a region outside the each of training images in which the lumen is present; estimate, based on the CNN, category information including a lumen direction of a region outside the captured image in which the lumen is likely to be present; and output the category information estimated.

In some embodiments, provided is an image processing method implemented by an image processing device including a processor. The image processing method includes: inputting a captured image captured by an endoscope in a body cavity of a subject to a Convolutional Neural Network (CNN) using a trained model, the trained model having training data in which each of training images is associated with category information that includes a lumen direction of a region outside the each of training images in which the lumen is present; estimating, based on the CNN, category information including a lumen direction of a region outside the captured image in which the lumen is likely to be present; and outputting the category information estimated.

In some embodiments, provided is a non-transitory computer readable recording medium having recorded therein an executable program. The program causes a computer to perform: inputting a captured image captured by an endoscope in a body cavity of a subject to a Convolutional Neural Network (CNN) using a trained model, the trained model having training data in which each of training images is associated with a category information that includes a lumen direction of a region outside the each of training images in which the lumen is present; estimating, based on the CNN, category information including a lumen direction of a region outside the captured image in which the lumen is likely to be present; and outputting the category information estimated.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of a medical system according to a first embodiment;

FIG. 2 is a block diagram illustrating a functional configuration of a main part of an endoscope and a control device according to the first embodiment;

FIG. 3 is a diagram illustrating input and output of a CNN using a trained model according to the first embodiment;

FIG. 4 is a flowchart illustrating an overview of a process that is performed by the control device according to the first embodiment;

FIG. 5 is a diagram schematically illustrating category information that is output, as an estimation result, by an estimation unit according to the first embodiment;

FIG. 6 is a diagram illustrating an example of an image that is displayed by a display device according to the first embodiment;

FIG. 7 is a diagram illustrating an example of an image that is displayed by a display device according to a first modification of the first embodiment;

FIG. 8 is a diagram illustrating an example of an image that is displayed by a display device according to a second modification of the first embodiment;

FIG. 9 is a diagram illustrating an example of an image that is displayed by a display device according to a third modification of the first embodiment;

FIG. 10 is a flowchart illustrating an overview of a process that is performed by a control device according to a fourth modification of the first embodiment;

FIG. 11 is a flowchart illustrating an overview of a process that is performed by a control device according to a fifth modification of the first embodiment;

FIG. 12 is a diagram illustrating an example of an image that is displayed by a display device according to a fifth modification of the first embodiment;

FIG. 13 is a diagram illustrating another example of the image that is displayed by the display device according to the fifth modification of the first embodiment;

FIG. 14 is a diagram illustrating still another example of the image that is displayed by the display device according to the fifth modification of the first embodiment;

FIG. 15 is a diagram illustrating still another example of the image that is displayed by the display device according to the fifth modification of the first embodiment;

FIG. 16 is a block diagram illustrating a functional configuration of a main part of an endoscope and a control device according to a second embodiment;

FIG. 17 is a flowchart illustrating an overview of a process that is performed by the control device according to the second embodiment;

FIG. 18 is a block diagram illustrating a functional configuration of a main part of an endoscope and a control device according to a third embodiment;

FIG. 19 is a diagram for explaining an overview of a data flow of a main part in a control unit according to the third embodiment;

FIG. 20 is a diagram for explaining a functional configuration of a coordinate transformation matrix calculation processing unit and an overview of a data flow of a coordinate transformation matrix calculation process according to the third embodiment;

FIG. 21 is a diagram for schematically explaining an overview of template matching that is performed by a template matching unit according to the third embodiment;

FIG. 22 is a flowchart illustrating an overview of a process that is performed by the control device according to the third embodiment;

FIG. 23 is a flowchart illustrating an overview of the coordinate transformation matrix calculation process in FIG. 22;

FIG. 24 is a flowchart illustrating an overview of a lumen coordinates calculation process in FIG. 22;

FIG. 25 is a diagram for schematically explaining an example of coordinates calculated by a lumen coordinates calculation unit according to the third embodiment;

FIG. 26 is a diagram illustrating another example of a direction category included in category information according to the third embodiment;

FIG. 27 is a diagram illustrating still another example of the direction category included in the category information according to the third embodiment;

FIG. 28 is a diagram illustrating still another example of the direction category included in the category information according to the third embodiment;

FIG. 29 is a diagram illustrating still another example of the direction category included in the category information according to the third embodiment;

FIG. 30 is a diagram illustrating still another example of the direction category included in the category information according to the third embodiment;

FIG. 31 is a block diagram illustrating a functional configuration of a main part of an endoscope and a control device according to a fourth embodiment;

FIG. 32 s a diagram for explaining an overview of a data flow of a main part in a control unit according to the fourth embodiment;

FIG. 33 is a flowchart illustrating an overview of a process that is performed by the control device according to the fourth embodiment;

FIG. 34 is a flowchart illustrating an overview of a lumen coordinates calculation process in FIG. 33;

FIG. 35 is a block diagram illustrating a functional configuration of a main part of an endoscope and a control device according to a fifth embodiment; and

FIG. 36 is a flowchart illustrating an overview of a process that is performed by the control device according to the fifth embodiment.

### DETAILED DESCRIPTION

A medical system according to the present disclosure will be described in detail below together with the drawings. The present disclosure is not limited by the embodiments below. Further, in each of the drawings to be referred to in the description below, shapes, sizes, and positional relationships are only schematically illustrated so as to make it possible to understand details of the present disclosure. In other words, the present disclosure is not limited to only the shapes, the sizes, and the positional relationships that are illustrated in each of the drawings. Furthermore, in the description of the drawings, explanation will be given by denoting the same components by the same reference symbols.

### First Embodiment

Overall configuration of medical system

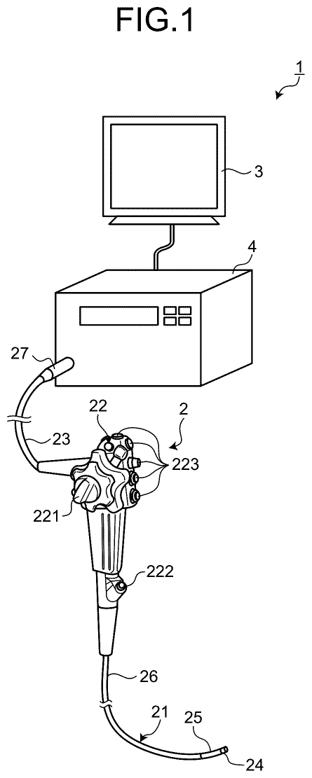

FIG. 1 is a diagram illustrating an overall configuration of a medical system according to a first embodiment. A medical system 1 illustrated in FIG. 1 successively captures images inside a subject, such as a human being or an animal, by inserting an insertion portion 21 of an endoscope 2 from a mouth to an esophagus of the subject or from an anus to a large intestine of the subject, outputs pieces of captured image data that are chronologically successive to a control device 4, and displays the pieces of captured image data on a display device 3. An operator, such as a doctor, performs observation and treatment on the subject while checking a display image that is displayed on the display device 3 and examination information that is output from the control device 4.

As illustrated in FIG. 1, the medical system 1 includes the endoscope 2, the display device 3, and the control device 4.

The endoscope 2 successively generates image data (RAW data) by capturing image inside the subject, and sequentially outputs the image data to the control device 4. As illustrated in FIG. 1, the endoscope 2 includes the insertion portion 21, an operating unit 22, and a universal cord 23.

The insertion portion 21 is configured such that at least a part of which has flexibility, and is inserted into a subject. As illustrated in FIG. 1, the insertion portion 21 includes a distal end portion 24 that is arranged on a distal end of the insertion portion 21, a bending portion 25 that is connected to a proximal end side (at the side of the operating unit 22) of the distal end portion 24 and that is configured in a bendable manner, and a flexible tube portion 26 that is connected to a proximal end side of the bending portion 25, that has flexibility, and that has an elongated shape.

The operating unit 22 is connected to a proximal end portion of the insertion portion 21. The operating unit 22 receives various kinds of operation on the endoscope 2. As illustrated in FIG. 1, the operating unit 22 includes a bending knob 221, an insertion opening 222, and a plurality of operating members 223.

The bending knob 221 is configured so as to be rotatable in accordance with user operation that is performed by a user, such as an operator. Further, the bending knob 221 rotates to cause a curved mechanism (not illustrated), such as a wire made of metal or resin, that is arranged inside the insertion portion 21. With this configuration, the bending portion 25 is curved.

The insertion opening 222 is an insertion opening that communicates with a treatment tool channel (not illustrated) that is a pipe extending from the distal end of the insertion portion 21, and that is used for inserting a treatment tool or the like into the treatment tool channel from outside of the endoscope 2.

The plurality of operating members 223 include buttons for receiving various kinds of operation that is performed by a user, such as an operator, and outputs an operation signal corresponding to each kind of operation to the control device 4 via the universal cord 23. Examples of various kinds of operation include release operation for instructing the endoscope 2 to capture a still image and operation of changing an observation mode of the endoscope 2 to a normal light observation mode or a special light observation mode.

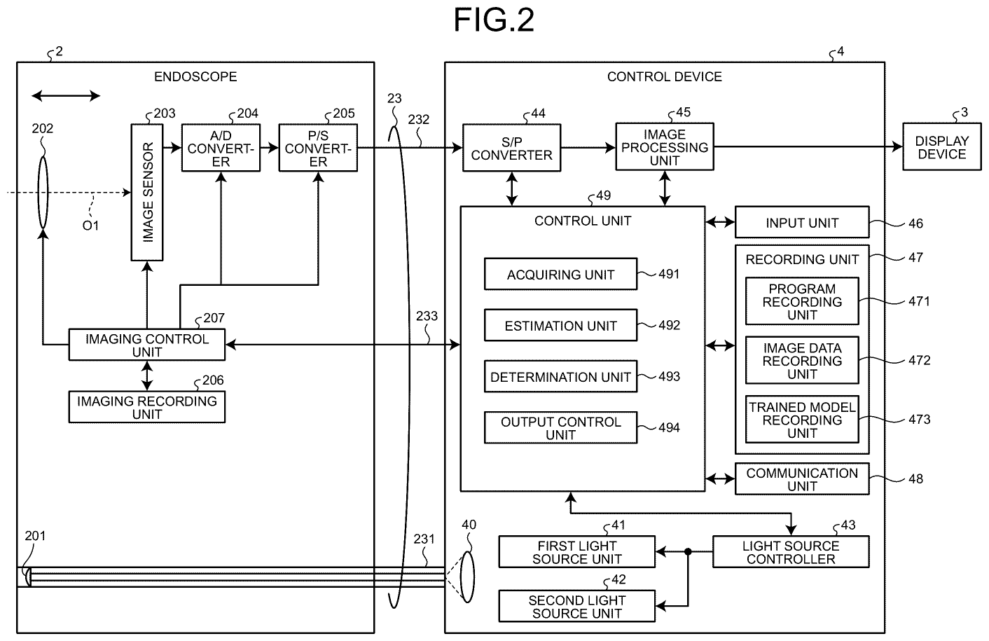

The universal cord 23 is a cord which extends from the operating unit 22 in a different direction from an extending direction of the insertion portion 21 and in which a light guide 231 (see FIG. 2) that is configured with an optical fiber or the like, a first signal line 232 (see FIG. 2) that transfers the image data as described above, a second signal line 233 (see FIG. 2) that transfers the operation signal as described above, and the like are arranged. Further, as illustrated in FIG. 1, a first connector 27 is arranged at a proximal end of the universal cord 23. The first connector 27 is irremovably connected to the control device 4.

The display device 3 is configured with a display monitor, such as liquid crystal or organic Electro Luminescence (EL), and displays a display image based on image data that is subjected to image processing by the control device and various kinds of information on the endoscope 2, under the control of the control device 4.

The control device 4 is realized by using a processor that is a processing apparatus including hardware, such as a Graphics Processing Unit (GPU), a Field Programmable Gate Array (FPGA), or a Central Processing Unit (CPU), and a memory that is a temporary storage area used by the processor. The control device 4 comprehensively controls operation of each of the units of the endoscope 2 in accordance with a program that is recorded in the memory.

### Functional Configuration of Main Part of Medical System

A functional configuration of a main part of the medical system 1 as described above will be described. FIG. 2 is a block diagram illustrating a functional configuration of a main part of the endoscope 2 and the control device 4. In the following, the endoscope 2 and the control device 4 will be described in this order.

### Functional Configuration of Endoscope

A configuration of the endoscope 2 will be described below.

As illustrated in FIG. 2, the endoscope 2 includes an illumination optical system 201, an imaging optical system 202, an image sensor 203, an A/D converter 204, a P/S converter 205, an imaging recording unit 206, and an imaging control unit 207. Here, each of the illumination optical system 201, the imaging optical system 202, the image sensor 203, the A/D converter 204, the P/S converter 205, the imaging recording unit 206, and the imaging control unit 207 is arranged in the distal end portion 24.

The illumination optical system 201 is configured with one or more lenses or the like, and emits illumination light that is supplied from the light guide 231 toward an imaging object.

The imaging optical system 202 is configured with an actuator that includes a stepping motor or a voice coil motor that moves a plurality of lenses and a predetermined lens among the plurality of lenses in an optical axis direction. The imaging optical system 202 condenses light, such as reflected light that is reflected from an imaging object, returning light that comes from the imaging object, or fluorescence that is emitted by the imaging object, and forms an object image on a light receiving surface of the image sensor 203. Further, the imaging optical system 202 is able to change a focal distance (imaging magnification or magnification) and a focal position by moving a predetermined lens along an optical axis direction O1 under the control of the imaging control unit 207. In one embodiment, the imaging optical system 202 is able to change the imaging magnification to one time, 80 times, or 520 times. The imaging optical system 202 need not, of course, change the imaging magnification in a stepwise manner, but the imaging magnification may be changed in a continuous manner.

The image sensor 203 is configured with an image sensor, such as a Charge Coupled Device (CCD) or a Complementary Metal Oxide Semiconductor (CMOS), in which one of color filters that form Bayer arrangement (RGGB) is arranged on each of pixels that are arranged in a two matrix manner. The image sensor 203 receives the object image that is formed by the imaging optical system 202, and generates a captured image (analog signal) by performing photoelectric conversion, under the control of the imaging control unit 207. Meanwhile, in the present embodiment, the image sensor 203 may be configured by integrating an image sensor and a Time Of Flight (TOF) sensor that acquires imaging object distance information (hereinafter, described as depth map information) by a TOF method. The depth map information is information in which an imaging object distance from a position of the image sensor 203 (position of the distal end portion 24) to a corresponding position on an observation object that corresponds to a pixel position of a captured image is detected for each of pixel positions. Meanwhile, the configuration that generates the depth map information is not limited to the TOF sensor as described above, but it may be possible to adopt an image sensor that includes a phase difference sensor. In the following, the depth map information and the captured image are collectively described as image data. The image sensor 203 outputs the image data to the A/D converter 204.

The A/D converter 204 is configured with an A/D conversion circuit or the like. The A/D converter 204 performs an A/D conversion process on an analog image data that is input from the image sensor 203 and outputs the image data to the P/S converter 205, under the control of the imaging control unit 207.

The P/S converter 205 is configured with a P/S conversion circuit or the like, performs parallel-to-serial conversion on digital image data that is input from the A/D converter 204 and outputs the image data to the control device 4 via the first signal line 232, under the control of the imaging control unit 207.

Meanwhile, it may be possible to arrange, instead of the P/S converter 205, an E/O converter that converts image data to an optical signal and output the image data by the optical signal to the control device 4. Further, for example, it may be possible to transmit image data to the control device 4 by radio communication using Wireless Fidelity (Wi-Fi) (registered trademark) or the like.

The imaging recording unit 206 is configured with a non-volatile memory or a volatile memory, and records therein various kinds of information on the endoscope 2 (for example, pixel information of the image sensor 203). Further, the imaging recording unit 206 records therein various kinds of setting data and a control parameter that are transferred from the control device 4 via the second signal line 233.

The imaging control unit 207 is realized by using a Timing Generator (TG), a processor that is a processing apparatus including hardware, such as a CPU, and a memory that is a temporary storage area used by the processor. The imaging control unit 207 control operation of each of the image sensor 203, the A/D converter 204, and the P/S converter 205 based on the setting data that is received from the control device 4 via the second signal line 233.

### Configuration of Control Device

A configuration of the control device 4 will be described below.

As illustrated in FIG. 2, the control device 4 includes a condenser lens 40, a first light source unit 41, a second light source unit 42, a light source controller 43, an S/P converter 44, an image processing unit 45, an input unit 46, a recording unit 47, a communication unit 48, and a control unit 49.

The condenser lens 40 collects light that is emitted by each of the first light source unit 41 and the second light source unit 42, and outputs the light to the light guide 231.

The first light source unit 41 emits white light (normal light) that is visible light, and supplies the white light, as illumination light, to the light guide 231, under the control of the light source controller 43. The first light source unit 41 is configured with a collimator lens, a white Light Emitting Diode (LED) lamp, a driving driver, and the like. Meanwhile, as the first light source unit 41, it may be possible to cause a red LED lamp, a green LED lamp, and a blue LED lamp to simultaneously emit light and supply white light that is visible light. Further, the first light source unit 41 may be configured with a halogen lamp, a xenon lamp, or the like.

The second light source unit 42 emits special light with a predetermined wavelength band and supplies the special light, as the illumination light, to the light guide 231, under the control of the light source controller 43. Here, the special light is light that is used for Narrow band Imaging (NBI) using narrow band light including 390 to 445 nanometers (nm) and 530 to 550 nm. It is of course possible to adopt, as the special light, light of amber color (600 nm and 630 nm) that is used for Red dichromatic Imaging (RDI), apart from the narrow band light.

The light source controller 43 is realized by using a processor that is a processing apparatus including hardware, such as a CPU, and a memory that is a temporary storage area used by the processor. The light source controller 43 controls a light emission timing, a light emission time, and the like of each of the first light source unit 41 and the second light source unit 42 based on control data that is input from the control unit 49.

The S/P converter 44 performs serial-to-parallel conversion on image data that is received from the endoscope 2 via the first signal line 232 and outputs the image data to the image processing unit 45 under the control of the control unit 49. Meanwhile, when the endoscope 2 outputs the image data by an optical signal, it may be possible to arrange an O/E converter that converts an optical signal to an electrical signal, instead of the S/P converter 44. Further, when the endoscope 2 transmits the image data by radio communication, it may be possible to arrange a communication module that can receive a radio signal, instead of the S/P converter 44.

The image processing unit 45 is realized by using a processor including hardware, such as a GPU or an FPGA, and a memory that is a temporary storage area used by the processor. The image processing unit 45 performs predetermined image processing on image data that is parallel data input from the S/P converter 44 and outputs the image data to the display device 3 under the control of the control unit 49. Examples of the predetermined image processing include demosaic processing, white balance processing, gain adjustment processing, Y correction processing, and format conversion processing.

The input unit 46 is configured with a mouse, a foot switch, a keyboard, a button, a switch, a touch panel, and the like, receives user operation that is performed by a user, such as an operator, and outputs an operation signal corresponding to the user operation to the control unit 49.

The recording unit 47 is configured with a volatile memory, a non-volatile memory, a Solid State Drive (SSD), a Hard Disk Drive (HDD), a recording medium, such as a memory card, or the like. Further, the recording unit 47 records therein data including various kinds of parameters that are needed for operation of the control device 4 and the endoscope 2. Furthermore, the recording unit 47 includes a program recording unit 471 for recording various kinds of programs for operating the endoscope 2 and the control device 4, an image data recording unit 472 for recording an image file in which an image corresponding to image data is recorded, and a trained model recording unit 473 for recording a trained model. Details of the trained model will be described later.

The communication unit 48 transmits various kinds of information to an external server via a network N100, receives various kinds of information from the server, and outputs the various kinds of information to the control unit 49, under the control of the control unit 49. The communication unit 48 is configured with a communication module or the like.

The control unit 49 corresponds to a second processor according to the present disclosure. The control unit 49 is realized by using a second processor that is a processing apparatus including hardware, such as an FPGA or a CPU, and a memory that is a temporary storage area used by the processor. Further, the control unit 49 comprehensively control each of the units included in the endoscope 2 and the control device 4. The control unit 49 includes an acquiring unit 491, an estimation unit 492, a determination unit 493, and an output control unit 494. Meanwhile, in the first embodiment, the control unit 49 functions as an image processing device.

The acquiring unit 491 acquires a captured image that corresponds to the image data that is captured by the endoscope 2 via the S/P converter 44.

The estimation unit 492 estimates category information in the captured image based on the captured image that is acquired by the acquiring unit 491 and the trained model that is recorded in the trained model recording unit 473.

The determination unit 493 determines whether or not a lumen is present in the captured image based on the category information that is estimated by the estimation unit 492. The determination unit 493 determines whether or not the estimation unit 492 is able to estimate a lumen direction based on the category information that is estimated by the estimation unit 492. Here, the lumen may refer to the position at the back of the lumen. For example, when an endoscope is inserted into the colon to capture images, it is the innermost position of the lumen in relation to the direction of insertion of the endoscope Within a range of observation by endoscopy. The lumen may also refer to an abbreviated cylindrical shadowed area located at the innermost point on the image. The lumen may be a deep portion located distally relative to a proximal intestinal wall.

The output control unit 494 causes the image processing unit 45 to superimpose lumen information that corresponds to the category information that is estimated by the estimation unit 492 onto a captured image that corresponds to captured image data that is generated by performing image processing by the image processing unit 45, and outputs the captured image with the lumen information to the display device 3.

### Overview of Trained Model

An overview of generation of the trained model that is recorded in the trained model recording unit 473 and estimation by a CNN using the trained model will be described below. The trained model is a parameter of the CNN, and, the trained model is generated, in advance, by training using training data and used as a parameter, so that the CNN is able to perform estimation on an image that is different from training data.

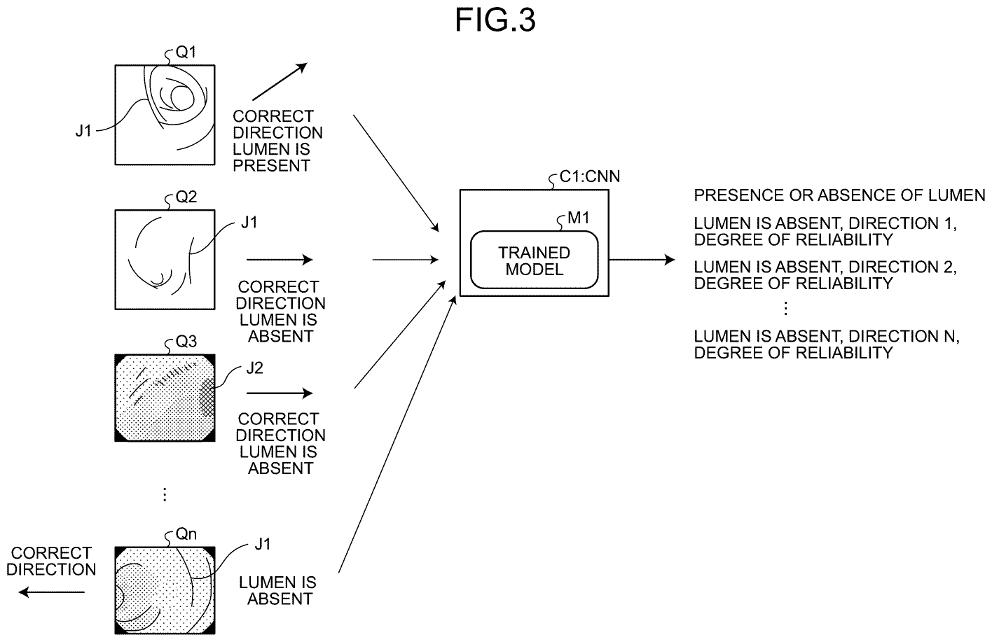

FIG. 3 is a diagram illustrating input and output of a CNN using a trained model M1. The CNN using the trained model M1 receives input of image data that is obtained by capturing an image inside a subject by a medical device, such as the endoscope 2, and outputs category information that includes presence or absence of a lumen in the captured image and, in the case where a lumen is absent, a lumen direction 1 to a lumen direction N (N=integer), each of which indicates a position of a lumen that is estimated to be present outside the captured image when directions viewed from the center of the image are divided by a predetermined angle, and a degree of reliability of each of the lumen directions. Here, the degree of reliability is a probability of the estimation result, such as a numerical value that can be represented by %, for example. The trained model is trained such that, as illustrated in FIG. 3, a plurality of training images Q1 to Qn (n=an integer equal to or larger than 4) each including at least one of a lumen, a wall J1 of an intestinal wall, and a shade J2 of a lumen of the subject can be associated with presence or absence of a lumen and a lumen direction indicating a position of the lumen from the center of the image, which are given as a correct value, by estimation by the CNN.

### Process Performed by Control Device

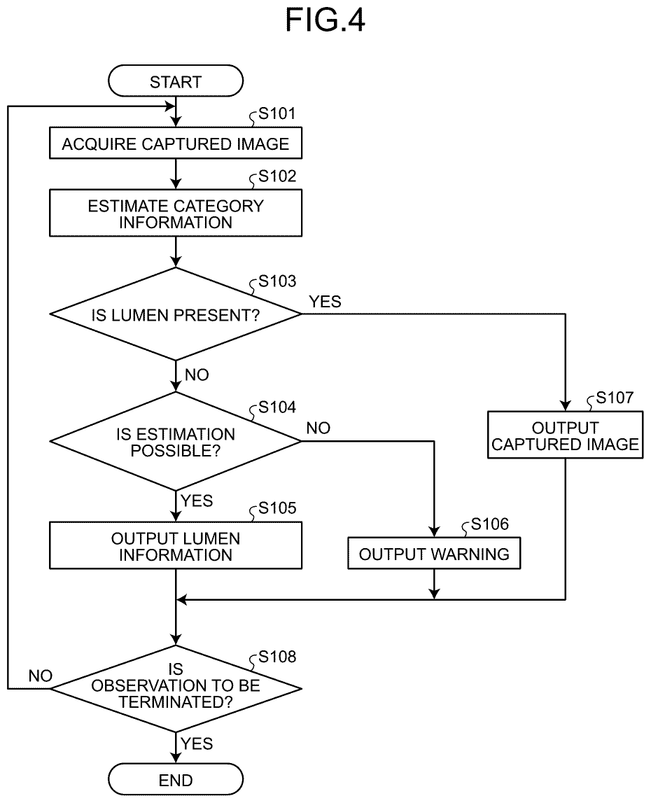

A process performed by the control device 4 will be described below. FIG. 4 is a flowchart illustrating an overview of a process that is performed by the control device 4.

At Step S101 illustrated in FIG. 4, first, the acquiring unit 491 of the control device 4 acquires a captured image that corresponds to the image data that is captured by the endoscope 2, via the S/P converter 44.

Subsequently, at Step S102, the estimation unit 492 estimates category information in the captured image from the captured image that is acquired by the acquiring unit 491, by the CNN using the trained model that is recorded in the trained model recording unit 473. Specifically, the estimation unit 492 inputs the captured image to the CNN using the trained model that is recorded in the trained model recording unit 473, and causes the category information that includes presence or absence of a lumen in the captured image to be output and, in the case where a lumen is absent, a lumen direction indicating a position of a lumen that is estimated to be present outside the captured image when directions viewed from the center of the image are divided by a predetermined angle, and a degree of reliability of the lumen direction to be output.

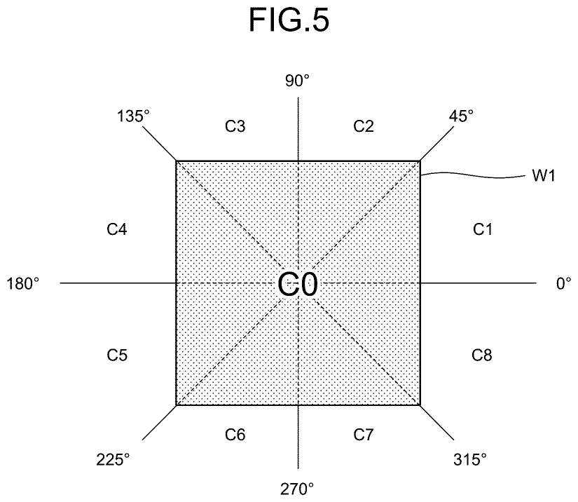

FIG. 5 is a diagram schematically illustrating the category information that is output, as an estimation result, by the estimation unit 492.

As illustrated in FIG. 5, the category information includes a category C0 indicating that a lumen is present in a captured image W1, a category C1 to a category C8 indicating that lumens are present in regions in directions outside the captured image W1 when the directions are divided by a predetermined angle, such as at intervals of 45 degrees, from the center of the captured image W1, and a degree of reliability of the estimation result of each of the category C0 to the category C8.

In this manner, the estimation unit 492 estimates the category information in the captured image from the captured image that is acquired by the acquiring unit 491 by the CNN using the trained model that is recorded in the trained model recording unit 473. Meanwhile, the estimation unit 492 outputs the category of each of the directions and the degree of reliability of each of the directions, but embodiments are not limited to this example, and it may be possible to output only the lumen direction with the highest degree of reliability.

Referring back to FIG. 4, explanation of Step S103 and subsequent processes will be continued.

At Step S103, the determination unit 493 determines whether or not a lumen is present in the captured image based on the category information that is estimated by the estimation unit 492. Specifically, the determination unit 493 determines whether or not a lumen is present in the captured image based on presence or absence of a lumen included in the category information that is estimated by the estimation unit 492. When the determination unit 493 determines that a lumen is present in the captured image (Step S103: Yes), the control device 4 goes to Step S107 to be described later. In contrast, when the determination unit 493 determines that a lumen is absent in the captured image (Step S103: No), the control device 4 goes to Step S104 to be described below. At Step S103, the determination unit may determine whether a deep portion (deep area or deep region) exists in the captured image. The deep portion may be a substantially circular or oval. The deep portion may be relatively distant from an endoscope, so that the illumination from the endoscope hardly reaches. Thus, the deep portion may be a dark area or dark region. The area in which dark pixels are gathered is extracted as a lumen deep portion.

At Step S104, the determination unit 493 determines whether or not the estimation unit 492 is able to estimate a lumen direction based on the category information that is estimated by the estimation unit 492. Specifically, the determination unit 493 determines whether or not the degree of reliability of each of the lumen directions included in the category information that is estimated by the estimation unit 492 is equal to or larger than a threshold, and determines whether or not the estimation unit 492 is able to estimate the lumen direction by determining whether or not the lumen direction for which the degree of reliability is equal to or larger than the threshold is present. For example, the determination unit 493 determines that the estimation unit 492 is able to estimate the lumen direction when the degree of reliability of at least one of the lumen directions is equal to or larger than the threshold, and determines that the estimation unit 492 is not able to estimate the lumen direction when the degrees of reliability of all of the lumen directions are not equal to or larger than the threshold. When the determination unit 493 determines that the estimation unit 492 is able to estimate a lumen direction (Step S104: Yes), the control device 4 goes to Step S105 to be described later. In contrast, when the determination unit 493 determines that the estimation unit 492 is not able to estimate a lumen direction (Step S104: No), the control device 4 goes to Step S106 to be described later.

At Step S105, the output control unit 494 causes the image processing unit 45 to superimpose lumen information that corresponds to the category information that is estimated by the estimation unit 492 onto the captured image that corresponds to the captured image data that is generated by performing image processing by the image processing unit 45, and outputs the captured image data with the lumen information to the display device 3.

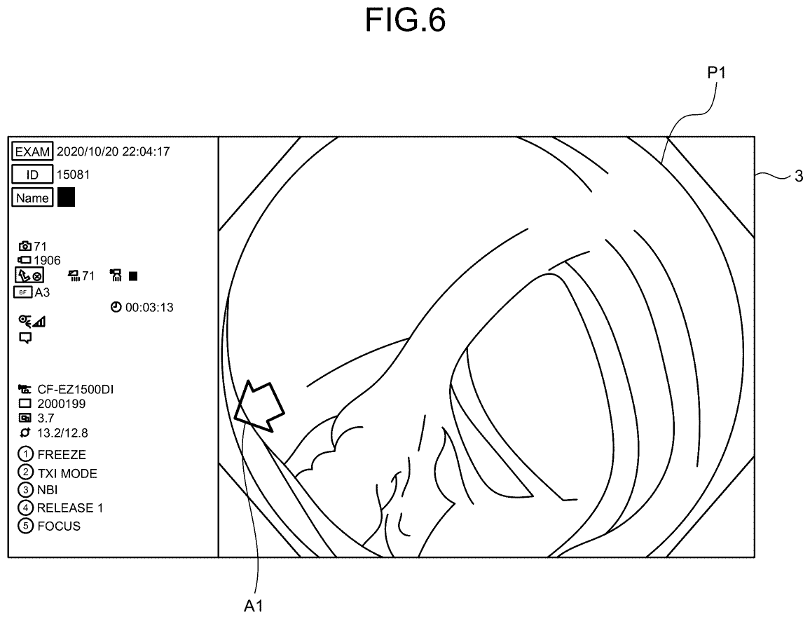

FIG. 6 is a diagram illustrating an example of an image that is displayed by the display device 3. As illustrated in FIG. 6, the output control unit 494 causes the image processing unit 45 to superimpose, as the lumen information, an arrow A1 corresponding to the category information that indicates a lumen direction outside a captured image P1 that is estimated by the estimation unit 492 onto the captured image P1 that corresponds to the captured image data that is generated by performing image processing by the image processing unit 45, and displays the captured image with the arrow on the display device 3. With this configuration, even when the captured image P1 does not include a lumen, an operator is able to intuitively recognize the lumen direction. After Step S105, the control device 4 goes to Step S108 to be described later.

At Step S106, the output control unit 494 causes the image processing unit 45 to superimpose a warning indicating that the estimation unit 492 fails to estimate the lumen direction onto a captured image that corresponds to the captured image data that is generated by performing image processing by the image processing unit 45, and outputs the captured image with the warning to the display device 3. For example, the output control unit 494 causes the image processing unit 45 to superimpose a message indicating that the lumen direction is not estimated or a message indicating that an image capturing direction of the insertion portion 21 of the endoscope 2 is to be changed onto the captured image, and displays the captured image with the message on the display device 3. Meanwhile, the output control unit 494 may outputs the warning by an alarm or a voice, instead of the message, to indicate that the estimation unit 492 fails to estimate the lumen direction. After Step S106, the control device 4 goes to Step S108 to be described later.

At Step S107, the output control unit 494 outputs the captured image that corresponds to the captured image data that is generated by performing image processing by the image processing unit 45 to the display device 3. After Step S107, the control device 4 goes to Step S108 to be described later.

At Step S108, the determination unit 493 determines whether or not the operator terminates observation of the subject. Specifically, the determination unit 493 determines whether or not a termination signal for terminating the observation of the subject is input from the operating unit 22 of the endoscope 2 by operation that is performed on the operating unit 22 of the endoscope 2 by the operator. When the determination unit 493 determines that the operator terminates the observation of the subject (Step S108: Yes), the control device 4 terminates the process. In contrast, when the determination unit 493 determines that the operator does not determine the observation of the subject (Step S108: No), the control device 4 returns to Step S101 as described above.

According to the first embodiment as described above, even when a lumen is not present in a captured image, it is possible to present a lumen direction.

Furthermore, according to the first embodiment, the output control unit 494 superimpose a warning indicating that the estimation unit 492 fails to estimate a lumen direction onto the captured image and outputs the captured image with the warning to the display device 3; therefore, when it is difficult to estimate a lumen, it is possible to actively encourage the operator to move the insertion portion 21 of the endoscope 2.

### First Modification of First Embodiment

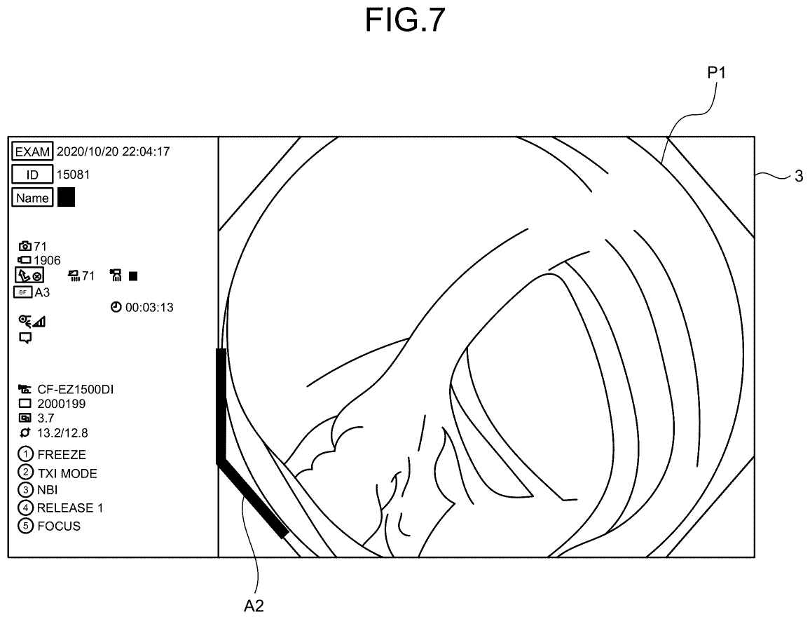

A first modification of the first embodiment will be described below. FIG. 7 is a diagram illustrating an example of an image that is displayed by the display device 3 according to the first modification of the first embodiment.

As illustrated in FIG. 7, the output control unit 494 causes the image processing unit 45 to superimpose, as the lumen information, a frame A2 corresponding to the category information that indicates a lumen direction outside the captured image P1 that is estimated by the estimation unit 492 onto the captured image P1 that corresponds to the captured image data that is generated by performing image processing by the image processing unit 45, and displays the captured image with the frame on the display device 3. Meanwhile, the output control unit 494 may display, in a highlighted manner, the frame A2 by highlight display or the like, or display the frame A2 with a changed color or a changed thickness.

According to the first modification of the first embodiment as described above, even when the captured image P1 does not include a lumen, an operator is able to intuitively recognize a lumen direction.

### Second Modification of First Embodiment

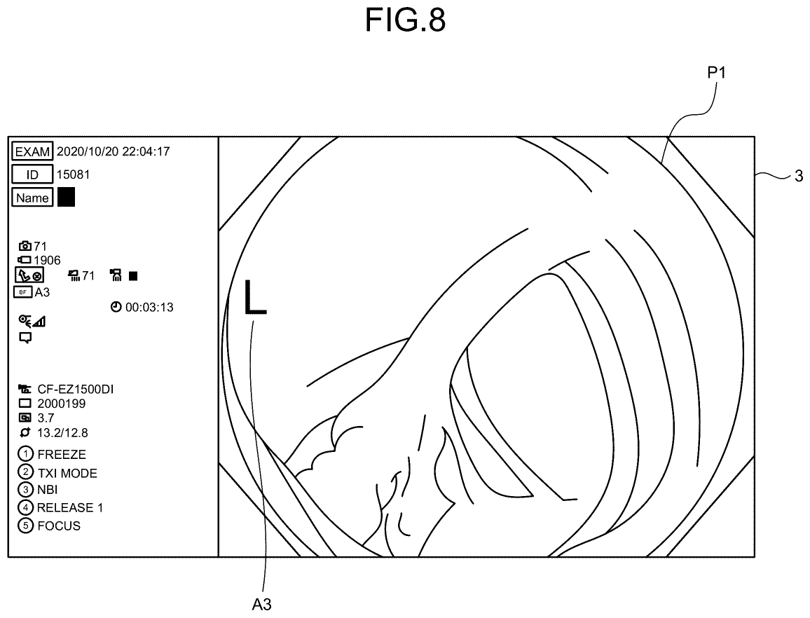

A second modification of the first embodiment will be described below. FIG. 8 is a diagram illustrating an example of an image that is displayed by the display device 3 according to the second modification of the first embodiment.

As illustrated in FIG. 8, the output control unit 494 causes the image processing unit 45 to superimpose, as the lumen information, a character A3 (L is displayed that represents a left side in the example in FIG. 8) corresponding to the category information that indicates a lumen direction outside the captured image P1 that is estimated by the estimation unit 492 onto the captured image P1 that corresponds to the captured image data that is generated by performing image processing by the image processing unit 45, and displays the captured image with the character on the display device 3. The character A3 may be displayed in a highlighted manner with a certain size that is changed in accordance with the degree of reliability.

According to the second modification of the first embodiment as described above, even when the captured image P1 does not include a lumen, an operator is able to intuitively recognize a lumen direction.

### Third Modification of First Embodiment

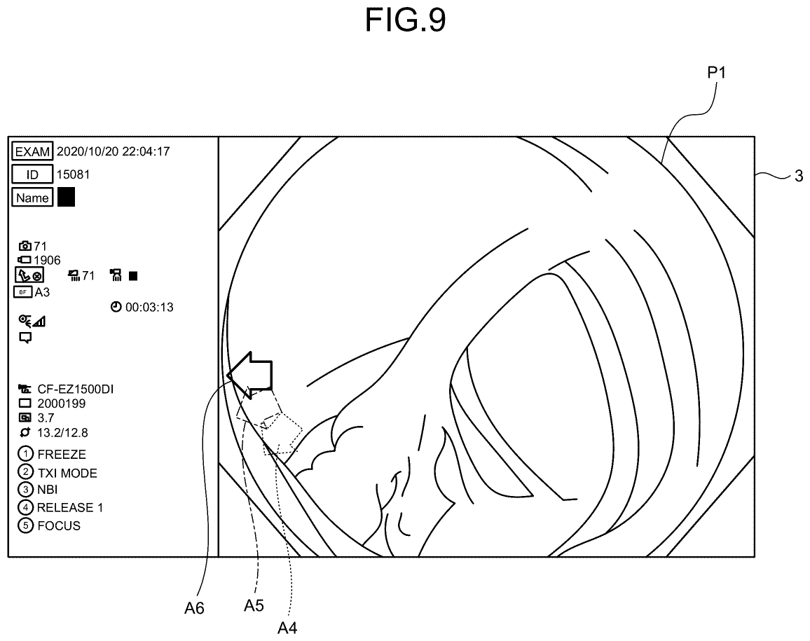

A third modification of the first embodiment will be described below. FIG. 9 is a diagram illustrating an example of an image that is displayed by the display device 3 according to the third modification of the first embodiment.

As illustrated in FIG. 9, the output control unit 494 causes the image processing unit 45 to superimpose an arrow A4 to an arrow A6 corresponding to the lumen information that indicates the lumen direction outside the captured image P1 that is estimated by the estimation unit 492 onto the captured image P1 that corresponds to the captured image data that is generated by performing image processing by the image processing unit 45, and displays the captured image with the arrows on the display device 3. The output control unit 494 simultaneously displays, as the lumen information, the arrow A4 to the arrow A6 corresponding to pieces of category information each of which is estimated in a past captured image that is a predetermined number of frames older than the latest captured image, on the display device 3. In this case, the output control unit 494 changes a display mode such that temporally older lumen information than a current time is displayed with lighter color, such that the colors of the arrow A6, the arrow A5, and the arrow A4 are gradually lightened in this order, and displays the arrows in the display mode on the display device 3, for example. It is of course possible for the output control unit 494 to change a display mode such that temporally older lumen information than a current time is displayed with lighter color, such that the colors of the arrow A6, the arrow A5, and the arrow A4 are gradually lightened in this order in accordance with details of operation that is performed on the operating unit 22 by the operator, and displays the arrows in the display mode on the display device 3, for example.

According to the third modification of the first embodiment as described above, even when the captured image P1 does not include a lumen, an operator is able to intuitively recognize a lumen direction and recognize that a lumen moves in accordance with operation on the endoscope 2.

### Fourth Modification of First Embodiment

A fourth modification of the first embodiment will be described below. In the fourth modification of the first embodiment, a part of the process executed by the control device 4 is different. Therefore, the process performed by the control device 4 will be described below.

### Process Performed by Control Device

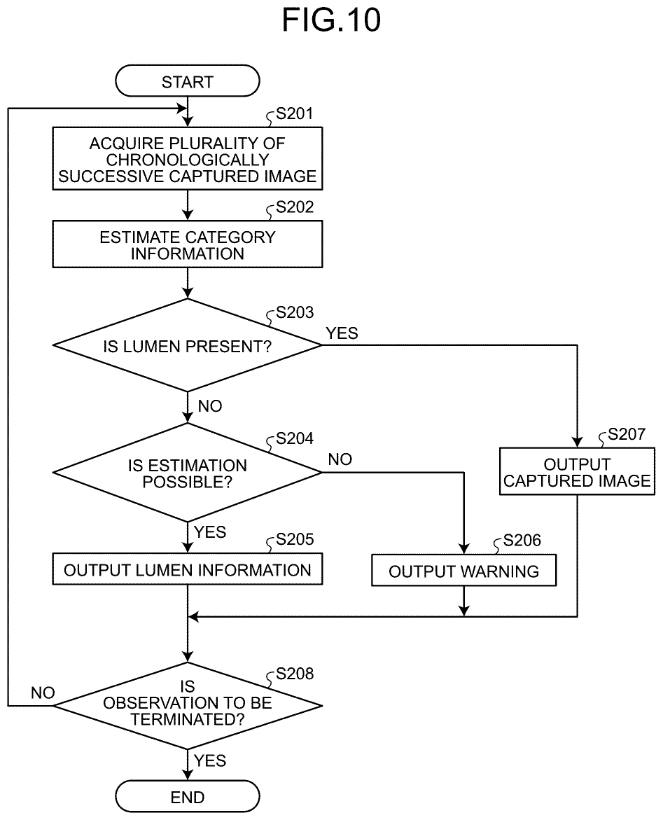

FIG. 10 is a flowchart illustrating an overview of a process that is performed by the control device 4 according to the fourth modification of the first embodiment. In FIG. 10, Step S203 to Step S208 respectively correspond to Step S103 to Step S108 in FIG. 4 as described above, and Step S201 and Step S202 are different. In the following, Step S201 and Step S202 performed by the control device 4 will be described.

At Step S201 illustrated in FIG. 10, the acquiring unit 491 acquires a plurality of captured images that are chronologically successive and that are captured by the endoscope 2 via the S/P converter 44.

Subsequently, the estimation unit 492 estimates the category information in the captured image based on the plurality of chronologically successive captured images that are acquired by the acquiring unit 491 and the trained model that is recorded in the trained model recording unit 473 (Step S202). Specifically, the estimation unit 492 inputs the plurality of chronologically successive captured images to the trained model, and causes the trained model to output the category information that includes presence or absence of a lumen in the captured image, the lumen direction, and the degree of reliability. In this case, the trained model may be generated by training using, as the CNN, a Long Short-Term Memory (LSTM) or the like. In this case, as the training data, it is satisfactory to use a plurality of chronologically successive captured images that are the plurality of captured images that are captured by the endoscope 2 in the subject. After Step S202, the control device 4 goes to Step S203.

According to the fourth modification of the first embodiment as described above, the estimation unit 492 inputs the plurality of chronologically successive captured images to the trained model, and causes the trained model to output the category information that includes presence or absence of a lumen in the captured image, the lumen direction, and the degree of reliability; therefore, it is possible to use a captured image without disturbance, such as a bubble, a residue in the subject, or blurring, and it is possible to use captured images that are captured in a plurality of different directions, so that it is possible to perform estimation with high accuracy.

### Fifth Modification of First Embodiment

A fifth modification of the first embodiment will be described below. In the fifth modification of the first embodiment, a part of the process executed by the control device 4 is different. Specifically, in the fifth modification of the first embodiment, a display mode is changed in accordance with the degree of reliability of the lumen direction included in the lumen information. Therefore, the process performed by the control device 4 will be described below.

### Process Performed by Control Device

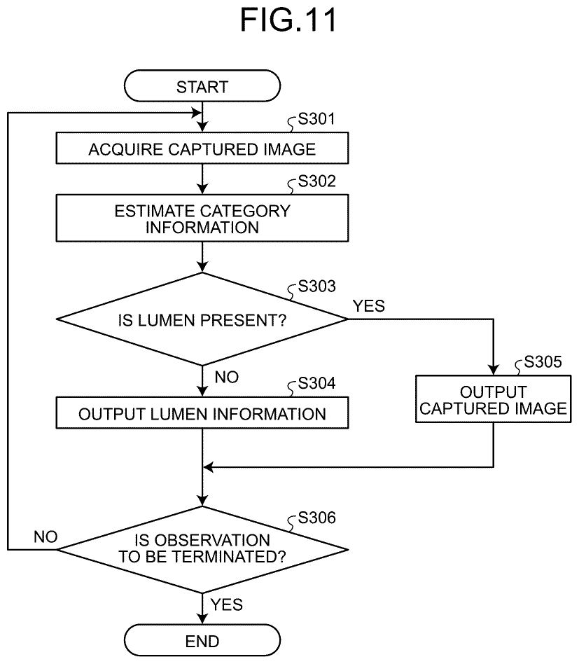

FIG. 11 is a flowchart illustrating an overview of a process that is performed by the control device 4 according to the fifth modification of the first embodiment. In FIG. 11, Step S301, Step S302, Step S305, and Step S306 respectively correspond to Step S101, Step S102, Step S107, and Step S108 in FIG. 4 as described above, and Step S303 and Step S304 are different. In the following, Step S303 and Step S304 performed by the control device 4 will be described.

At Step S303, the determination unit 493 determines whether or not a lumen is present in the captured image based on the lumen information that is estimated by the estimation unit 492. Specifically, the determination unit 493 determines whether or not a lumen is present in the captured image based on presence or absence of a lumen included in the category information that is estimated by the estimation unit 492. When the determination unit 493 determines that a lumen is present in the captured image (Step S303: Yes), the control device 4 goes to Step S305. In contrast, when the determination unit 493 determines that a lumen is absent in the captured image (Step S303: No), the control device 4 goes to Step S304 to be described later.

At Step S304, the output control unit 494 causes the image processing unit 45 to superimpose the lumen information that corresponds to the category information that is estimated by the estimation unit 492 onto a captured image that corresponds to the captured image data that is generated by performing image processing by the image processing unit 45, and outputs the captured image with the lumen information on the display device 3.

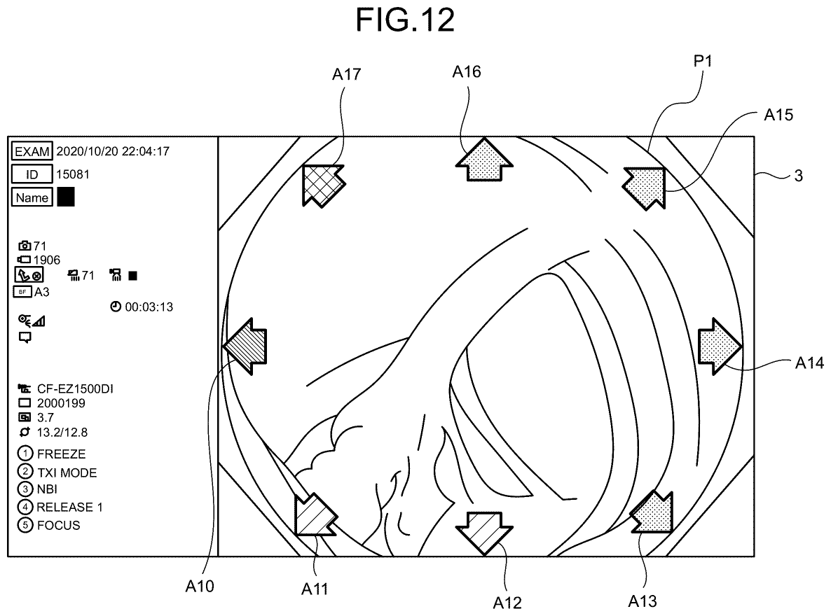

FIG. 12 is a diagram illustrating an example of the image that is displayed on the display device 3. As illustrated in FIG. 12, the output control unit 494 superimposes, as the lumen information, an arrow A10 to an arrow A17 for which display modes are changed in accordance with the degrees of reliability of the respective lumen directions included in the category information that is estimated by the estimation unit 492 onto the captured image P1, and displays the captured image with the arrows on the display device 3. With this configuration, even when the captured image P1 does not include a lumen, an operator is able to intuitively recognize a lumen direction.

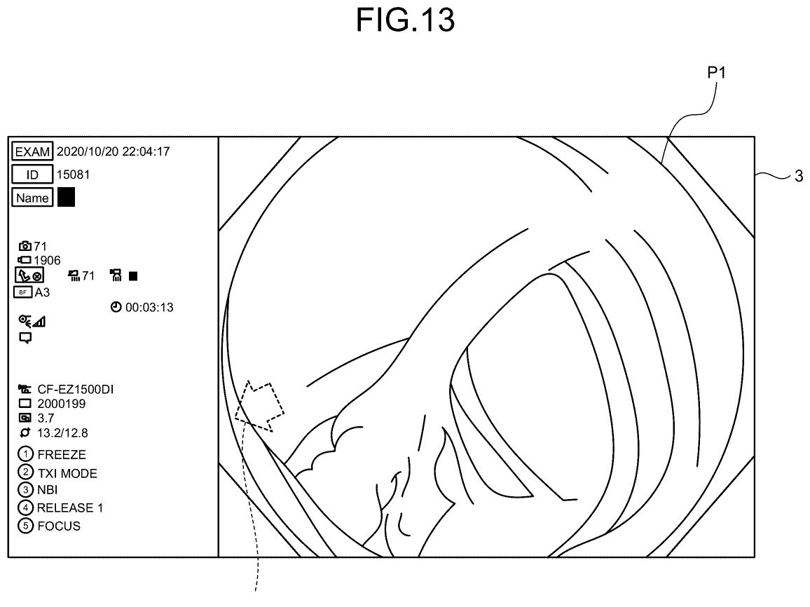

FIG. 13 is a diagram illustrating another example of the image that is displayed by the display device 3. As illustrated in FIG. 13, the output control unit 494 may superimpose, as the lumen information in the captured image P1, an arrow A21 corresponding to the lumen direction and the degree of reliability in the category information onto a lumen direction with the highest degree of reliability among the degrees of reliability of the plurality of lumen directions included in the category information that is estimated by the estimation unit 492, and display the captured image with the arrow on the display device 3. Here, a color and a density of the lumen are changed in accordance with the degree of reliability. With this configuration, even when the captured image P1 does not include a lumen, an operator is able to intuitively recognize a lumen direction and recognize a probability of estimation of the lumen direction.

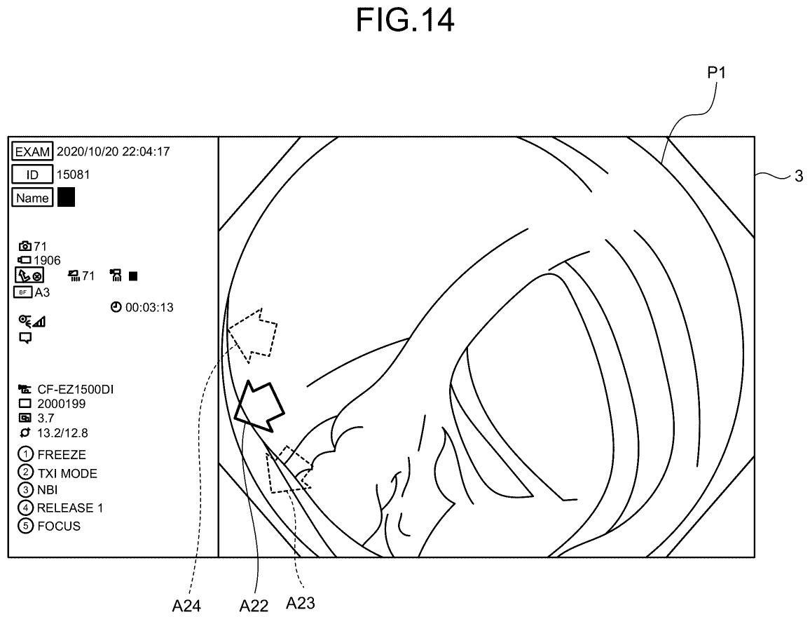

FIG. 14 is a diagram illustrating another example of the image that is displayed by the display device 3. As illustrated in FIG. 14, the output control unit 494 may superimpose, as pieces of lumen information in the captured image P1, a plurality of arrows A22 to A24 corresponding a plurality of the lumen directions and the degrees of reliability in the category information onto the plurality of lumen directions for which the degrees of reliability are high among the degrees of reliability of the plurality of lumen directions included in the category information that is estimated by the estimation unit 492, and display the captured image with the arrows on the display device 3. Here, a color and a density of the lumen are changed in accordance with the degree of reliability. With this configuration, even when the captured image P1 does not include a lumen, an operator is able to intuitively recognize a lumen direction and recognize a probability of estimation of the lumen direction.

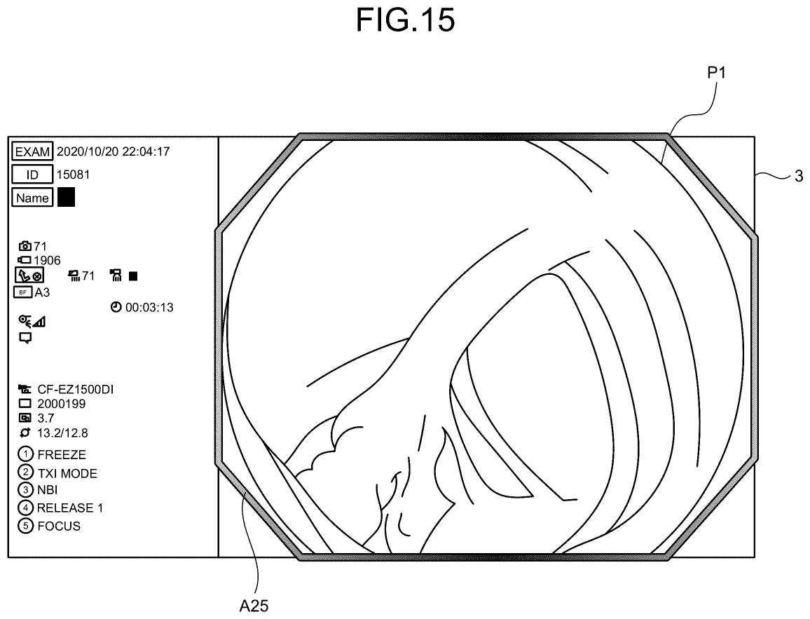

FIG. 15 is a diagram illustrating another example of the image that is displayed by the display device 3. As illustrated in FIG. 15, the output control unit 494 may change a display mode of a frame A25, as the lumen information, around the captured image P1 in accordance with the degrees of reliability of the plurality of lumen directions included in the lumen information that is estimated by the estimation unit 492, and display the frame in the changed display mode on the display device 3. In this case, the output control unit 494 changes a color of the frame A25 in accordance with the degree of reliability such that the color is changed in a gradation manner from blue to red with an increase in the degree of reliability, and displays the frame in the gradation manner on the display device 3. With this configuration, even when the captured image P1 does not include a lumen, an operator is able to intuitively recognize a lumen direction and recognize a probability of estimation of the lumen direction.

According to the fifth modification of the first embodiment as described above, even when the captured image P1 does not include a lumen, an operator is able to intuitively recognize a lumen direction and recognize a probability of estimation of the lumen direction.

### Second Embodiment

A second embodiment will be described below. In the second embodiment, a region of a subject that is captured by the endoscope 2 is estimated based on the captured image, a trained model corresponding to the region is selected from among a plurality of trained models based on the estimation result of the region, and estimation is performed. In the following, a functional configuration of a control device according to the second embodiment will be first described, and thereafter, a process performed by the control device according to the second embodiment will be described.

### Functional Configuration of Control Device

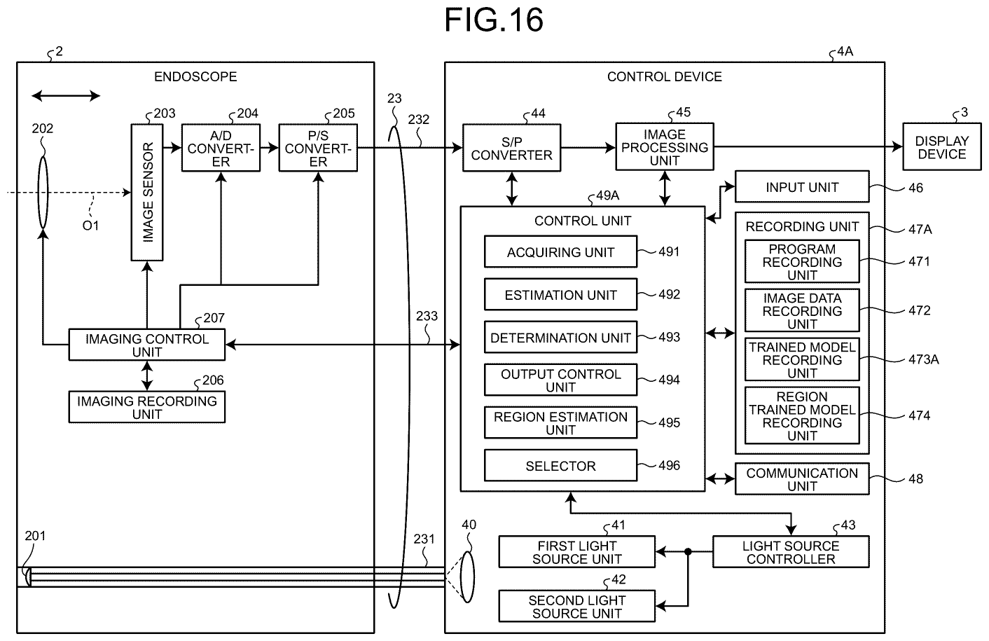

FIG. 16 is a block diagram illustrating a functional configuration of a main part of an endoscope and a control device according to the second embodiment. A control device 4A illustrated in FIG. 16 includes a recording unit 47A and a control unit 49A, instead of the recording unit 47 and the control unit 49 of the control device 4 according to the first embodiment as described above.

The recording unit 47A includes a trained model recording unit 473A, instead of the trained model recording unit 473 of the recording unit 47 according to the first embodiment as described above. Further, the recording unit 47A includes a region trained model recording unit 474.

The trained model recording unit 473A records therein a plurality of trained models that are able to estimate lumen information for each organ or each region in the subject. Specifically, the trained model recording unit 473A records therein a plurality of trained models corresponding to recto sigmoid, sigmoid colon, descending colon, transverse colon, ascending colon, upper rectum, and lower rectum. The plurality of trained models as described above has performed training for each organ or each region by training data in which the CNN described in the first embodiment, a plurality of pieces of image data, each of which is captured for each organ or each region and includes at least one of a lumen, a wall of an intestinal wall, and a shade of a lumen of the subject, and a lumen direction as a correct value are associated with one another.

The region trained model recording unit 474 records therein a region trained model for estimating a region of the subject that is captured by the endoscope 2. Specifically, the region trained model has performed training on training data in which the CNN described above in the first embodiment, a plurality of pieces of image data that are captured for each organ or each region, and a name of the organ or the region as a correct value are associated with one another. The region trained model receives input of the captured image data and outputs the name of the organ or the region.

The control unit 49A further includes a region estimation unit 495 and a selector 496 in addition to the functional configuration of the control unit 49 according to the first embodiment as described above.

The region estimation unit 495 estimates, from the captured image that is acquired by the acquiring unit 491, a region of the subject that is captured by the endoscope 2 by the CNN using the region trained model that is recorded in the region trained model recording unit 474.

The selector 496 selects a trained model corresponding to the region of the subject from among a plurality of trained models that are recorded in the trained model recording unit 473A based on the region of the subject that is estimated by the region estimation unit 495.

### Process Performed by Control Device

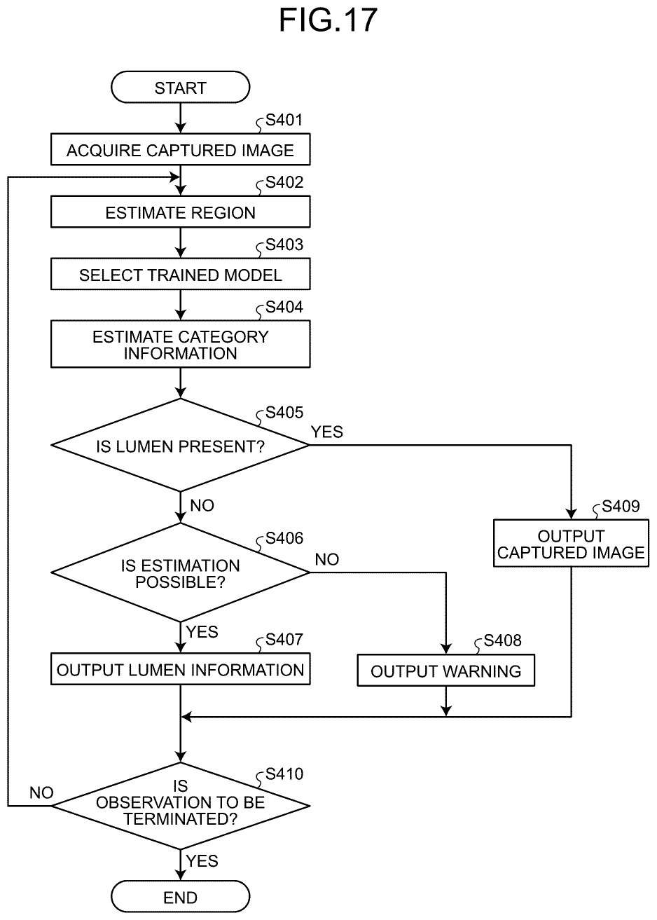

A process performed by the control device 4A will be described below. FIG. 17 is a flowchart illustrating an overview of a process that is performed by the control device 4A. In FIG. 17, Step S401 and Step S405 to Step S410 respectively correspond to Step S101 and Step S103 to Step S108 in FIG. 4 as described above, and Step S402 to Step S404 are different. In the following, Step S402 to Step S404 performed by the control device 4A will be described.

At Step S402, the region estimation unit 495 estimates a region of the subject that is captured by the endoscope 2 from the captured image that is acquired by the acquiring unit 491 by the CNN using the region trained model that is recorded in the region trained model recording unit 474 (Step S402). Specifically, the region estimation unit 495 inputs the captured image to the CNN using the region trained model, and causes a name of the region of the subject to be output.

Subsequently, the selector 496 selects a trained model that corresponds to the region of the subject from among a plurality of trained models that are recorded in the trained model recording unit 473A, based on the region of the subject that is estimated by the region estimation unit 495 (Step S403).

Thereafter, the estimation unit 492 estimates the lumen information in the captured image from the captured image that is acquired by the acquiring unit 491 by the CNN using the trained model that is selected by the selector 496 (Step S404). Specifically, the estimation unit 492 inputs the captured image to the CNN using the trained model that is selected by the selector 496, and causes the category information that includes presence or absence of a lumen in the captured image, a lumen direction, and a degree of reliability to be output. After Step S404, the control device 4A goes to Step S405.

According to the second embodiment as described above, the estimation unit 492 estimates the category information in the captured image from the captured image that is acquired by the acquiring unit 491 by the CNN using the trained model that is selected by the selector 496, so that it is possible to estimate the category information by using the trained model that is suitable for the region that is currently captured by the endoscope 2.

Meanwhile, in the second embodiment, the selector 496 selects the trained model that corresponds to the region of the subject from among the plurality of trained models that are recorded in the trained model recording unit 473A based on the organ or the region that is estimated by the region estimation unit 495, but embodiments are not limited to this example, and it may be possible to select a trained model that corresponds to an instruction signal that designates an organ or a region and that is input from the operating unit 22 or the input unit 46, from among the plurality of trained models that are recorded in the trained model recording unit 473A, for example.

### Third Embodiment

A third embodiment will be described below. In the third embodiment, coordinates of an observation position in the captured image are identified, and lumen information is displayed by using an identification result. In the following, a functional configuration of a control device according to the third embodiment will be first described, and thereafter, a process performed by the control device according to the third embodiment will be described.

### Functional Configuration of Control Device

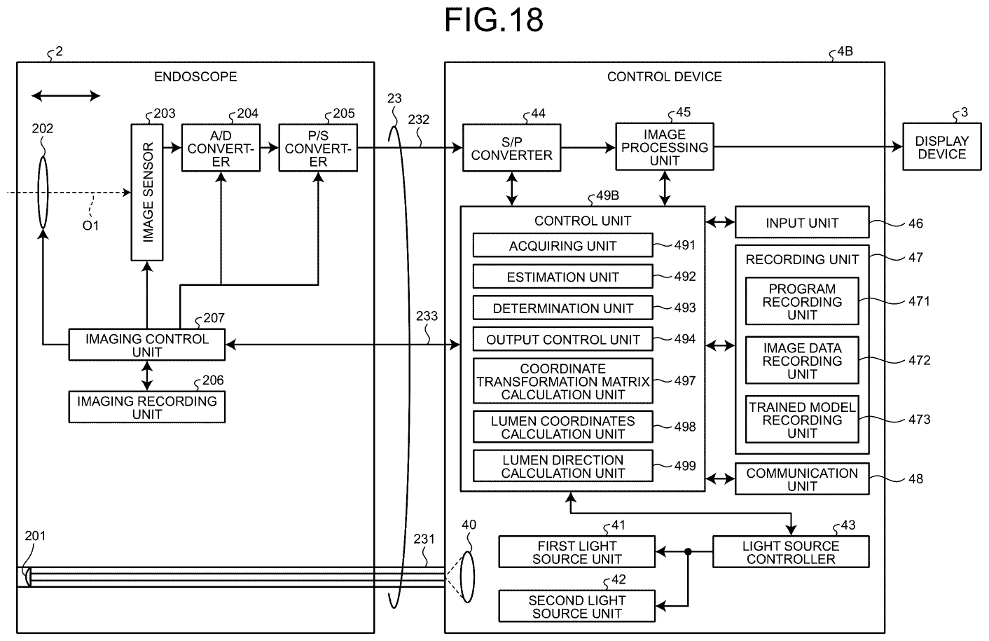

FIG. 18 is a block diagram illustrating a functional configuration of a main part of an endoscope and a control device according to the third embodiment. A control device 4B illustrated in FIG. 18 includes a control unit 49B, instead of the control unit 49 of the control device 4 according to the first embodiment as described above. The control unit 49B further includes a coordinate transformation matrix calculation unit 497, a lumen coordinates calculation unit 498, and a lumen direction calculation unit 499, in addition to the functional configuration of the control unit 49 according to the first embodiment as described above.

The coordinate transformation matrix calculation unit 497 calculates an affine matrix by performing a coordinate transformation matrix calculation process by using the captured image P1, determines trackability of lumen tracking, and outputs results to the lumen coordinates calculation unit 498.

The lumen coordinates calculation unit 498 calculates lumen coordinates of a lumen in the captured image P1 based on the category information that is input from the estimation unit 492 and the affine matrix and the trackability of the lumen tracking that are input from the coordinate transformation matrix calculation unit 497, outputs the lumen coordinates to the lumen direction calculation unit 499, and outputs a possibility of estimation of a lumen to the determination unit 493.

The lumen direction calculation unit 499 calculates a lumen direction based on the lumen coordinates that are input from the lumen coordinates calculation unit 498, and outputs the lumen direction to the output control unit 494.

### Overview of Data Flow of Main Part of Control Unit

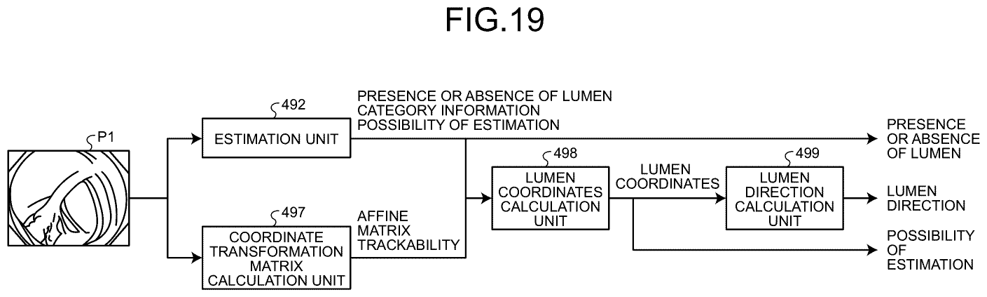

An overview of a data flow of a main part in the control unit 49B will be described below. FIG. 19 is a diagram for explaining the overview of the data flow of the main part in the control unit 49B.

As illustrated in FIG. 19, first, the control unit 49B inputs the captured image P1 to the estimation unit 492 and the coordinate transformation matrix calculation unit 497.

Subsequently, the estimation unit 492 estimates, from the captured image P1, the category information by using the CNN, and when presence or absence of a lumen that is the estimated category information indicates that a lumen is absent, the estimation unit 492 outputs the category information that includes the lumen direction 1 to the lumen direction N, the degree of reliability of each of the directions, and a possibility of estimation of a lumen to the lumen coordinates calculation unit 498 and the determination unit 493.

Further, the coordinate transformation matrix calculation unit 497 performs the coordinate transformation matrix calculation process by using the captured image P and outputs the affine matrix and the trackability of the lumen tracking to the lumen coordinates calculation unit 498, at the same time with the estimation process that is performed by the estimation unit 492. Meanwhile, details of the coordinate transformation matrix calculation process performed by the coordinate transformation matrix calculation unit 497 will be described later.

Subsequently, the lumen coordinates calculation unit 498 calculates the lumen coordinates of the lumen in the captured image P1 based on the category information that is input from the estimation unit 492 and the affine matrix and the trackability of the lumen tracking that are input from the coordinate transformation matrix calculation unit 497, outputs the lumen coordinates to the lumen direction calculation unit 499, and outputs the possibility of estimation of a lumen to the determination unit 493. Meanwhile, the lumen coordinates calculation process that are calculated by the lumen coordinates calculation unit 498 will be described later.

Thereafter, the lumen direction calculation unit 499 calculates and outputs the lumen direction based on the lumen coordinates that are input from the lumen coordinates calculation unit 498.

### Functional Configuration and Overview of Data Flow of Coordinate Transformation Matrix Calculation Unit

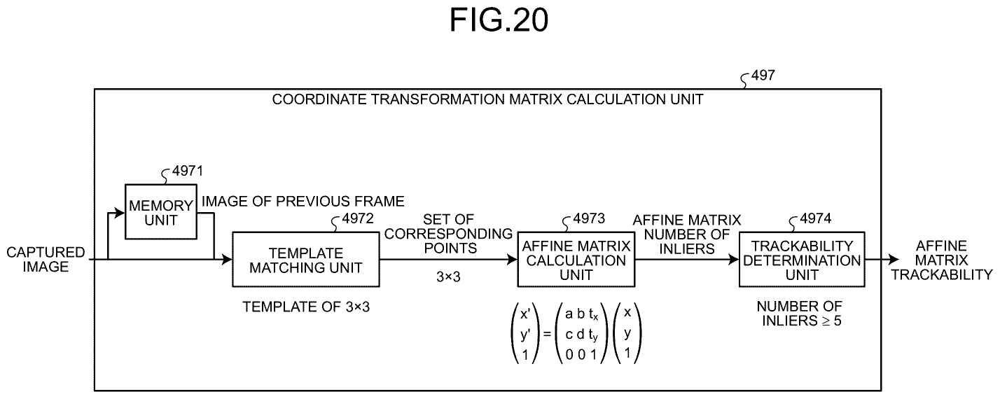

A functional configuration and the coordinate transformation matrix calculation process of the coordinate transformation matrix calculation unit 497 will be described below. FIG. 20 is a diagram for explaining the functional configuration and the overview of the data flow of the coordinate transformation matrix calculation process of the coordinate transformation matrix calculation unit 497.

As illustrated in FIG. 20, the coordinate transformation matrix calculation unit 497 includes a memory unit 4971, a template matching unit 4972, an affine matrix calculation unit 4973, and a trackability determination unit 4974.

The memory unit 4971 temporarily records therein the input captured image until next frame processing. Further, the memory unit 4971 outputs a captured image of a previous frame to the template matching unit 4972. The memory unit 4971 is configured with, for example, a frame memory or the like.

The template matching unit 4972 performs template matching between the captured image of the previous frame recorded in the memory unit 4971 and a current captured image.

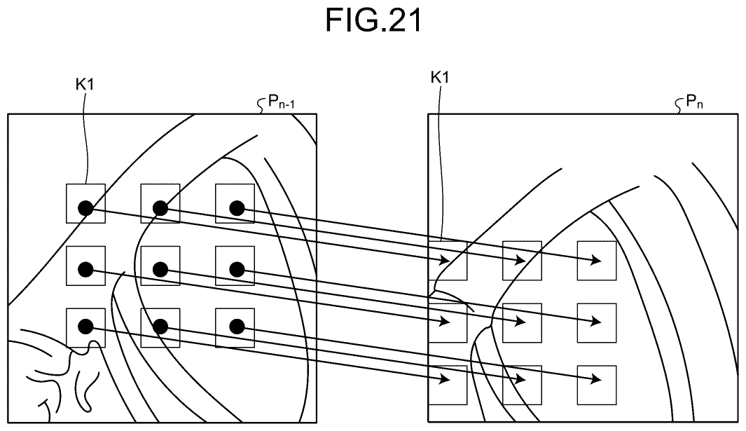

FIG. 21 is a diagram for schematically explaining an overview of the template matching performed by the template matching unit 4972. As illustrated in FIG. 21, the template matching unit 4972 performs a template matching process between a captured image Pₙ₋₁ of the previous frame and a captured image Pₙ of a current frame. Specifically, the template matching unit 4972 generates a set of nine corresponding points between the captured image Pₙ₋₁ of the previous frame and the captured image Pₙ of the current frame by using a template K1 of 3×3, and outputs the nine corresponding points to the affine matrix calculation unit 4973.

Referring back to FIG. 20, explanation of the functional configuration of the coordinate transformation matrix calculation unit 497 will be continued.

The affine matrix calculation unit 4973 performs an affine matrix calculation process based on the set of corresponding points input from the template matching unit 4972, and outputs an affine matrix and the number of inliers to the trackability determination unit 4974. Specifically, the affine matrix calculation unit 4973 performs a process as described below.

The affine matrix calculation unit 4973 randomly extracts n corresponding points that are minimum corresponding points needed for estimation of a parameter from the set of corresponding points input from the template matching unit 4972 (Step A).

Subsequently, the affine matrix calculation unit 4973 estimates a parameter by using a set of n corresponding points (Step B).

Thereafter, the affine matrix calculation unit 4973 assigns all of points other than the points that are extracted at Step A as described above to the parameter that is estimated at Step B, and compares an error between data that is obtained by the assignment and original data (Step C).

Further, the affine matrix calculation unit 4973 determines whether the error that is calculated at Step C is equal to or smaller than a threshold t, and if the error is equal to or smaller than the threshold t, the error is counted as an inlier (Step D).

Finally, the affine matrix calculation unit 4973 repeats Step A to Step D as described above, and extracts the affine matrix (a model parameter or a homography matrix) for which the number of inliers is the largest and the largest number of inliers (Step E).

The trackability determination unit 4974 determines whether or not the number of inliers that is input from the affine matrix calculation unit 4973 is equal to or larger than a threshold. Specifically, the trackability determination unit 4974 determines whether or not the number of inliers is equal to or larger than 5 (the number of inliers≥5). Thereafter, the trackability determination unit 4974 outputs the trackability based on the determination result of the number of inliers and the affine matrix that are input from the affine matrix calculation unit 4973 to the lumen coordinates calculation unit 498.

### Process Performed by Control Device

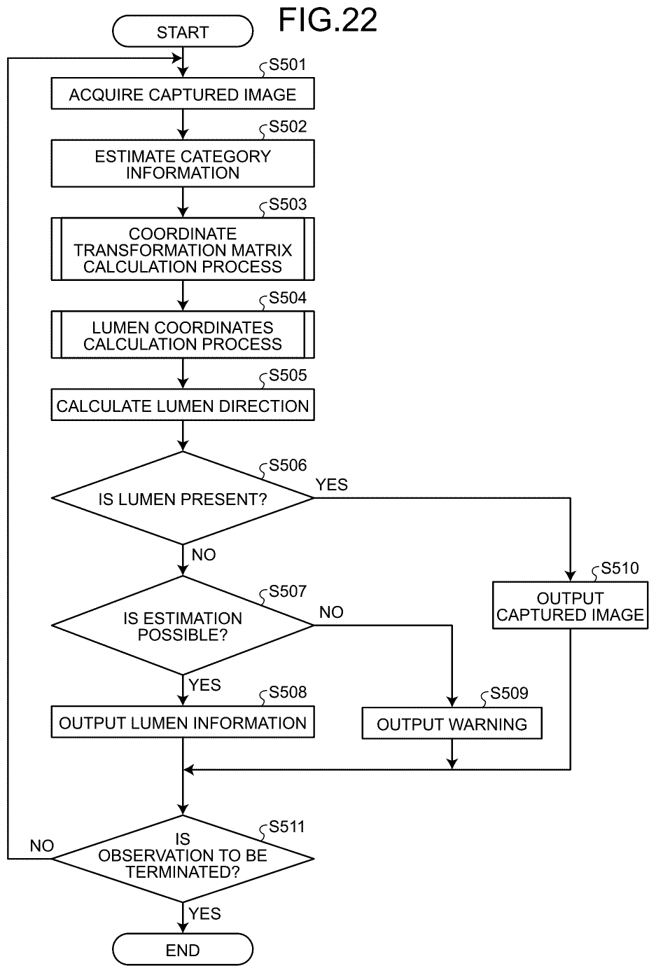

A process performed by the control device 4B will be described below. FIG. 22 is a flowchart illustrating an overview of a process that is performed by the control device 4B. Step S501, Step S502, and Step S506 to Step S511 respectively correspond to Step S101, Step S102, and Step S103 to Step S108 in FIG. 4 as described above, and Step S503 to Step S505 are different. In the following, Step S503 to Step S505 will be described.

At Step S503, the coordinate transformation matrix calculation unit 497 performs the coordinate transformation matrix calculation process by using the captured image P1 that is acquired by the acquiring unit 491.

### Overview of Coordinate Transformation Matrix Calculation Process

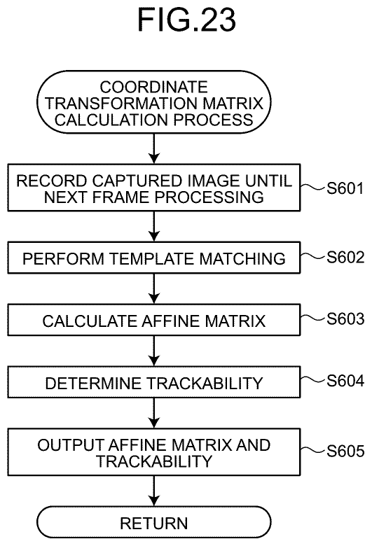

FIG. 23 is a flowchart illustrating an overview of the coordinate transformation matrix calculation process at Step S503.

At Step S601 illustrated in FIG. 23, first, the memory unit 4971 temporarily records therein the input captured image until next frame processing.

Subsequently, the template matching unit 4972 performs template matching between the captured image of the previous frame recorded in the memory unit 4971 and the current captured image (Step S602). Specifically, the template matching unit 4972 performs the template matching as described above, generates a set of nine corresponding points between the captured image Pₙ₋₁ of the previous frame and the captured image Pₙ of the current frame, and outputs the set of nine corresponding points to the affine matrix calculation unit 4973.

Thereafter, the affine matrix calculation unit 4973 performs the affine matrix calculation process based on the set of corresponding points input from the template matching unit 4972 and calculates the affine matrix and the number of inliers (Step S603). Specifically, the affine matrix calculation unit 4973 executes Step A to Step E as described above, and calculates the affine matrix (a model parameter or a homography matrix) for which the number of inliers is the largest and the largest number of inliers.

Subsequently, the trackability determination unit 4974 determines whether or not the number of inliers that is input from the affine matrix calculation unit 4973 is equal to or larger than a threshold (Step S604). Specifically, the trackability determination unit 4974 determines whether or not the number of inliers is equal to or larger than 5 (the number of inliers≥5).

Thereafter, the trackability determination unit 4974 outputs the trackability based on the determination result of the number of inliers and the affine matrix that are input from the affine matrix calculation unit 4973 to the lumen coordinates calculation unit 498 (Step S605). After Step S605, the control device 4 returns to the main routine in FIG. 22 and goes to Step S504.

Referring back to FIG. 22, explanation of Step S504 and subsequent processes will be continued.

At Step S504, the lumen coordinates calculation unit 498 performs a lumen coordinates calculation process of calculating the lumen coordinates of the lumen in the captured image P1 based on the category information that is input from the estimation unit 492 and the affine matrix and the trackability of the lumen tracking that are input from the coordinate transformation matrix calculation unit 497, outputting the lumen coordinates to the lumen direction calculation unit 499, and outputting the possibility of estimation of a lumen to the determination unit 493.

### Overview of Lumen Coordinates Calculation Process

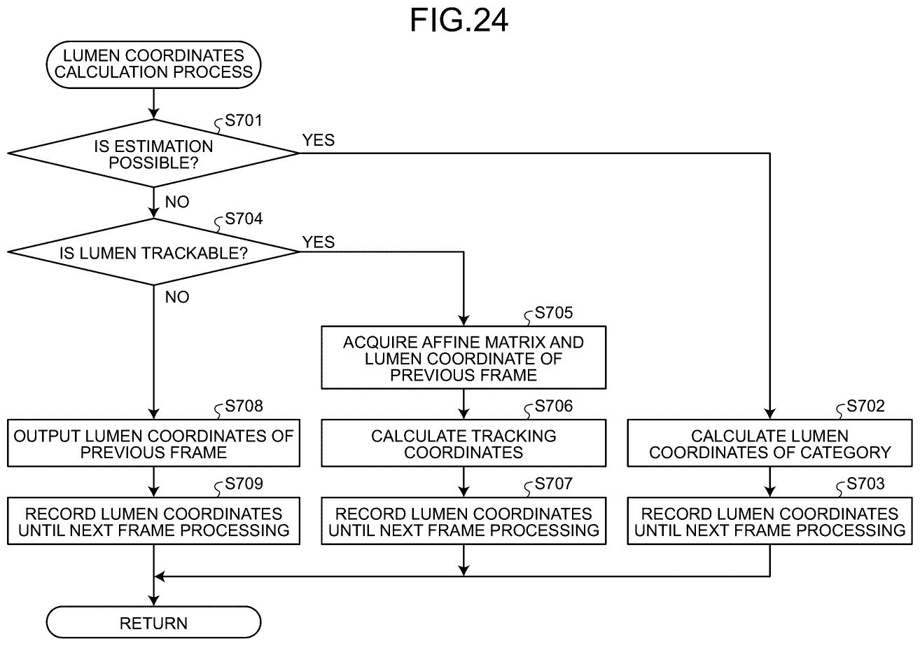

FIG. 24 is a flowchart illustrating an overview of the lumen coordinates calculation process at Step S504.

At Step S701 illustrated in FIG. 24, first, the lumen coordinates calculation unit 498 determines whether or not it is possible to estimate a lumen based on the category information that is input from the estimation unit 492. When the lumen coordinates calculation unit 498 determines that it is possible to estimate a lumen (Step S701: Yes), the control device 4B goes to Step S702 to be described later. In contrast, when the lumen coordinates calculation unit 498 determines that it is difficult to estimate a lumen (Step S701: No), the control device 4B goes to Step S704 to be described later.

At Step S702, the lumen coordinates calculation unit 498 calculates coordinates of the category of the lumen.

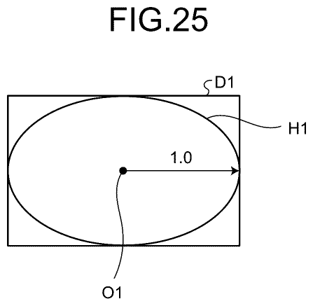

FIG. 25 is a diagram for schematically explaining an example of coordinates calculated by the lumen coordinates calculation unit 498. As illustrated in FIG. 25, when the captured image D1 does not include a lumen, the lumen coordinates calculation unit 498 assumes that a lumen is present in the lumen direction that is included in the category information that is input from the estimation unit 492, calculates, as the coordinates of the lumen, values that are obtained by multiplying a distance from a center O1 of the captured image D1 to an end portion (1. 0) by a predetermined value, for example, by 1.2, and obtains the coordinates of the category of the lumen. Meanwhile, the lumen coordinates calculation unit 498 may assume that a lumen is present in the lumen direction that is included in the category information that is input from the estimation unit 492, and calculate, as the coordinates of the lumen, values that are obtained by multiplying a predetermined region H1 in the captured image D1 by a predetermined value, for example, by 1.2. Furthermore, when the captured image D1 includes a lumen, the lumen coordinates calculation unit 498 calculates the center O1 of the captured image P1 as the coordinates of the lumen. Meanwhile, the lumen coordinates calculation unit 498 performs multiplication by 1.2 as a predetermined value, but embodiments are not limited to this example, and the predetermined value may be changed appropriately depending on an operator.

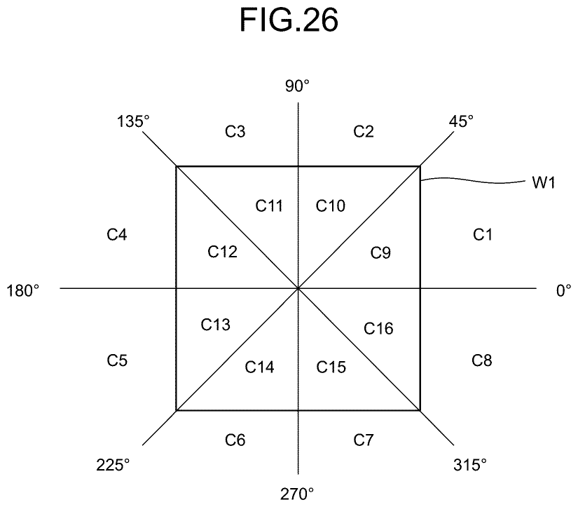

FIG. 26 is a diagram illustrating an example of a direction category that is included in the category information. As illustrated in FIG. 26, the category information further includes categories C9 to C16 each of which indicates a position of a lumen when the lumen is present in the captured image W1, in addition to the category C1 to the category C8 each of which indicates that a lumen is present in a region in a direction outside the captured image W1. Specifically, the categories C9 to C16 are set by dividing the captured image W1 by a predetermined angle, such as at intervals of 45 degrees, for example. With this configuration, when a lumen is present, it is possible to more accurately estimate lumen coordinates.

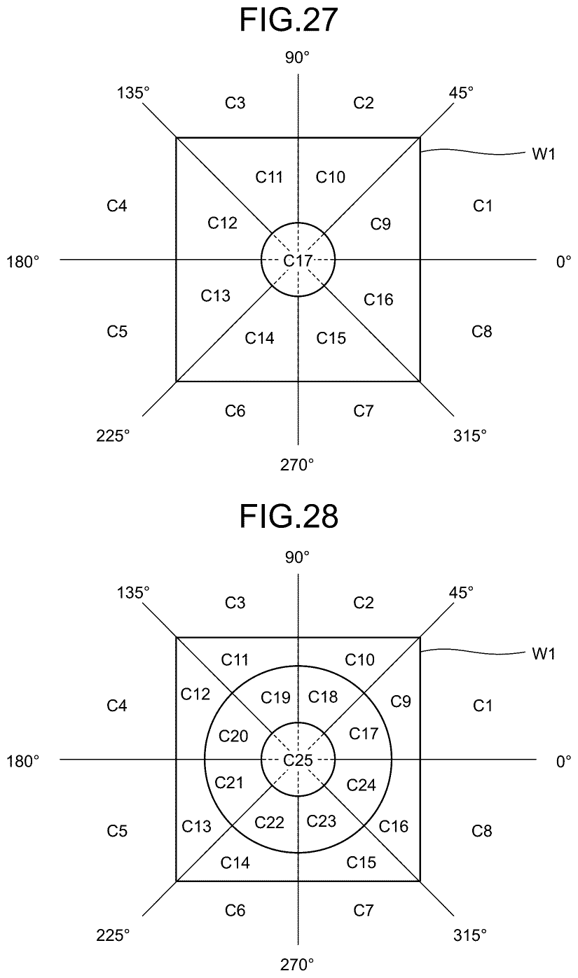

FIG. 27 is a diagram illustrating an example of the direction category that is included in the category information. As illustrated in FIG. 27, the category information further includes categories C9 to C17 each of which indicates a position of a lumen when the lumen is present in the captured image W1, in addition to the category C1 to the category C8 each of which indicates that a lumen is present in a region in a direction outside the captured image W1. The category C17 indicates a central region of the captured image W1. With this configuration, when a lumen is present, and even if the lumen is present in the central region of the captured image W1, it is possible to more accurately estimate the lumen coordinates.

FIG. 28 is a diagram illustrating an example of the direction category included in the category information. As illustrated in FIG. 28, the category information further includes categories C9 to C25 each of which indicates a position of a lumen when the lumen is present in the captured image W1, in addition to the category C1 to the category C8 each of which indicates that a lumen is present in a region in a direction outside the captured image W1. With this configuration, when a lumen is present, it is possible to more accurately perform estimation with respect to a difference in a distance of the lumen from the center of the captured image W1.

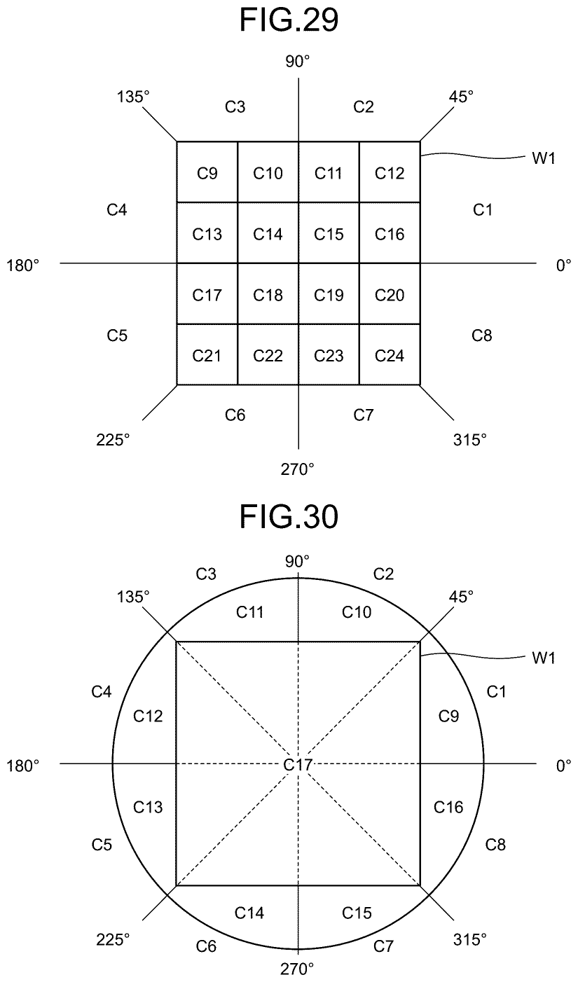

FIG. 29 is a diagram illustrating an example of the direction category included in the category information. As illustrated in FIG. 29, the category information further includes categories C9 to C24 each of which indicates a position of a lumen when the lumen is present in the captured image W1, in addition to the category C1 to the category C8 each of which indicates that a lumen is present in a region in a direction outside the captured image W1. With this configuration, when a lumen is present, it is possible to more accurately estimate a region in which the lumen is present among regions that are equally divided in a horizontal direction and a vertical direction in the captured image W1.

FIG. 30 is a diagram illustrating an example of the direction category included in the category information. As illustrated in FIG. 30, the category information includes the category C1 to the category C16 each of which indicates that a lumen is present in a region in a direction outside the captured image W1 and the category C17 which indicates a position of a lumen when the lumen is present in the captured image W1. Even when a lumen is absent, it is possible to more accurately estimate the lumen coordinates in accordance with a difference in the distance from the center of the image.

Referring back to FIG. 24, explanation of Step S703 and subsequent processes will be continued.

At Step S703, the lumen coordinates calculation unit 498 records therein the lumen coordinates calculated at Step S702 until next frame processing on the captured image. After Step S703, the control device 4B returns to the main routine in FIG. 22 and goes to Step S505.

At Step S704, the lumen coordinates calculation unit 498 determines whether or not a lumen is trackable in the captured image of the current frame based on the trackability that is input from the coordinate transformation matrix calculation unit 497. When the lumen coordinates calculation unit 498 determines that the lumen is trackable in the captured image of the current frame from the captured image of the previous frame (Step S704: Yes), the control device 4B goes to Step S705 to be described later. In contrast, when the lumen coordinates calculation unit 498 determines that the lumen is not trackable in the captured image of the current frame from the captured image of the previous frame (Step S704: No), the control device 4B goes to Step S708 to be described later.

At Step S705, the lumen coordinates calculation unit 498 acquires the affine matrix and the lumen coordinates of the previous frame that are input from the coordinate transformation matrix calculation unit 497.

Subsequently, the lumen coordinates calculation unit 498 calculates tracking coordinates for tracking the lumen in the captured image of the current frame based on the affine matrix and the lumen coordinates of the previous frame that are input from the coordinate transformation matrix calculation unit 497 (Step S706).

Thereafter, the lumen coordinates calculation unit 498 records the lumen coordinates calculated at Step S706 until next frame processing on the captured image (Step S707). After Step S707, the control device 4B returns to the main routine in FIG. 22 and goes to Step S505.

At Step S708, the lumen coordinates calculation unit 498 outputs the coordinates of the previous frame that are recorded at Step S703 or Step S707 as described above.

Subsequently, the lumen coordinates calculation unit 498 records the lumen coordinates that are output at Step S708 as described above (Step S709). After Step S709, the control device 4B returns to the main routine in FIG. 22.

Referring back to FIG. 22, explanation of Step S505 and subsequent processes will be continued.

At Step S505, the lumen direction calculation unit 499 calculates the lumen direction based on the lumen coordinates that are input from the lumen coordinates calculation unit 498, and outputs the lumen direction to the output control unit 494.

Subsequently, the determination unit 493 determines whether or not a lumen is present in the captured image based on the category information that is estimated by the estimation unit 492 (Step S506). Specifically, the determination unit 493 determines whether or not a lumen is present in the captured image based on presence or absence of a lumen that is included in the category information that is estimated by the estimation unit 492. When the determination unit 493 determines that a lumen is present in the captured image (Step S506: Yes), the control device 4B goes to Step S510. In contrast, when the determination unit 493 determines that a lumen is absent in the captured image (Step S506: No), the control device 4B goes to Step S507 to be described later.

At Step S507, the determination unit 493 determines whether or not the estimation unit 492 is able to estimate the lumen direction based on the category information that is estimated by the estimation unit 492. When the determination unit 493 determines that the estimation unit 492 is able to estimate the lumen direction (Step S507: Yes), the control device 4 goes to Step S508 to be described later. In contrast, when the determination unit 493 determines that the estimation unit 492 is not able to estimate the lumen direction (Step S507: No), the control device 4B goes to Step S509.

At Step S508, the output control unit 494 causes the image processing unit 45 to superimpose the lumen information that corresponds to the category information corresponding to the lumen direction that is calculated by the lumen direction calculation unit 499 onto the captured image that corresponds to the captured image data that is generated by performing image processing by the image processing unit 45, and output the captured image with the lumen information to the display device 3. After Step S508, the control device 4B goes to Step S511.

According to the third embodiment as described above, it is possible to output the lumen direction even when a lumen is absent in the captured image, and, when it is possible to track a corresponding point between chronologically successive captured images, it is possible to output the lumen direction with high accuracy.

### Fourth Embodiment

A fourth embodiment will be described below. In the fourth embodiment, a lumen in a captured image is detected based on the captured image. In the following, a functional configuration of a control device according to the fourth embodiment will be first described, and thereafter, a process performed by the control device according to the fourth embodiment will be described.

### Functional Configuration of Control Device

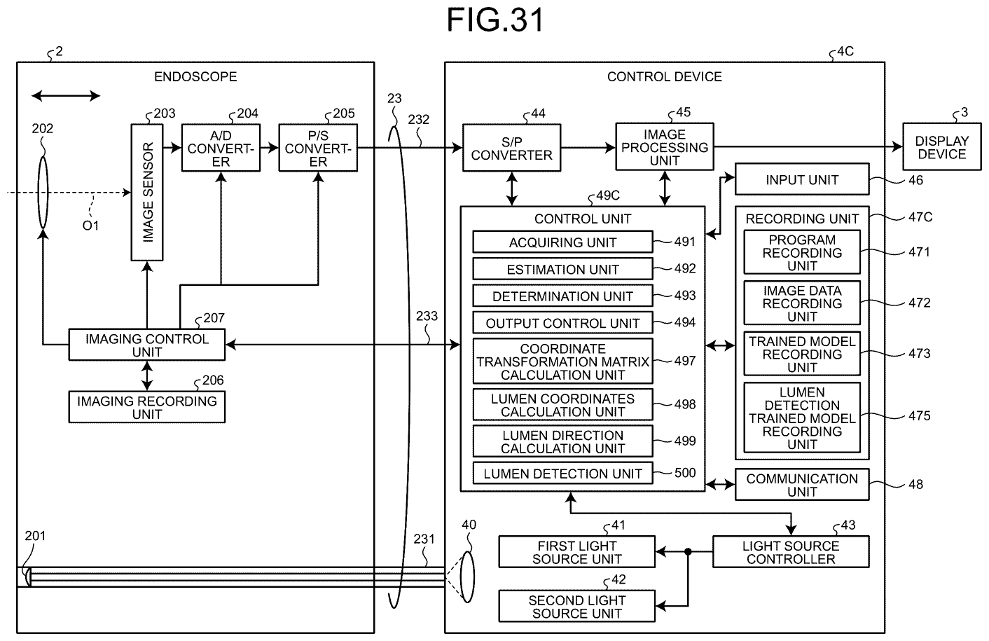

FIG. 31 is a block diagram illustrating a functional configuration of a main part of the endoscope and the control device according to the fourth embodiment. A control device 4C illustrated in FIG. 31 includes a recording unit 47C and a control unit 49C, instead of the recording unit 47 and the control unit 49B of the control device 4B according to the third embodiment as described above.

The recording unit 47C further includes a lumen detection trained model recording unit 475, in addition to the functional configuration of the recording unit 47 of the first embodiment as described above.

The lumen detection trained model recording unit 475 records therein a lumen detection trained model. The lumen detection trained model is for estimating presence or absence of a lumen in the captured image that is captured by the endoscope 2. Specifically, the lumen detection trained model is obtained by causing a CNN to perform training on training data in which each of training images that correspond to pieces of captured data that are captured by the endoscope 2 is associated with presence or absence of a lumen in each of the training images. The CNN using the lumen detection trained model receives input of the captured image data and outputs presence or absence of a lumen in the captured image.

The control unit 49C further includes a lumen detection unit 500, in addition to the functional configuration of the control unit 49B according to the third embodiment as described above.

The lumen detection unit 500 outputs presence or absence of a lumen in the captured image, from the captured image, by the CNN using the lumen detection trained model that is recorded in the lumen detection trained model recording unit 475, and outputs detected coordinates at which the lumen is detected when the lumen is present.

### Overview of Data Flow of Main Part of Control Unit

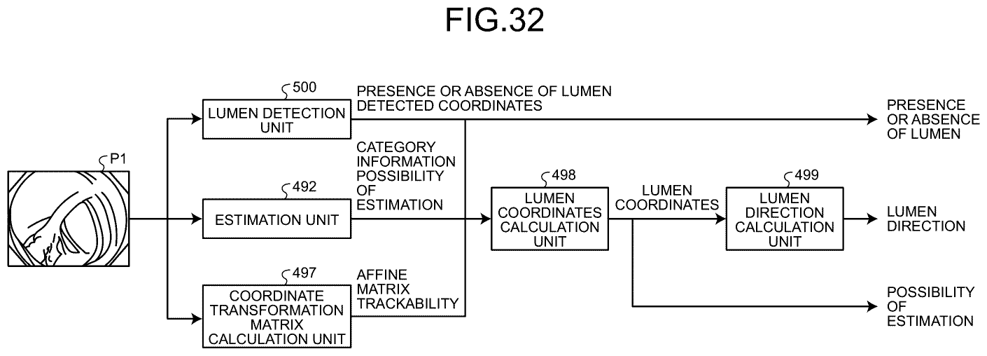

An overview of a data flow of a main part of the control unit 49C will be described below. FIG. 32 is a diagram for explaining an overview of the data flow of the main part of the control unit 49C.

As illustrated in FIG. 32, first, the control unit 49C inputs the captured image P1 to each of the lumen detection unit 500, the estimation unit 492, and the coordinate transformation matrix calculation unit 497. In this case, the lumen detection unit 500 outputs presence or absence of a lumen in the captured image P1, from the captured image P1, by the CNN using the lumen detection trained model that is recorded in the lumen detection trained model recording unit 475, and outputs detected coordinates at which the lumen is detected when the lumen is present.

### Process Performed by Control Device

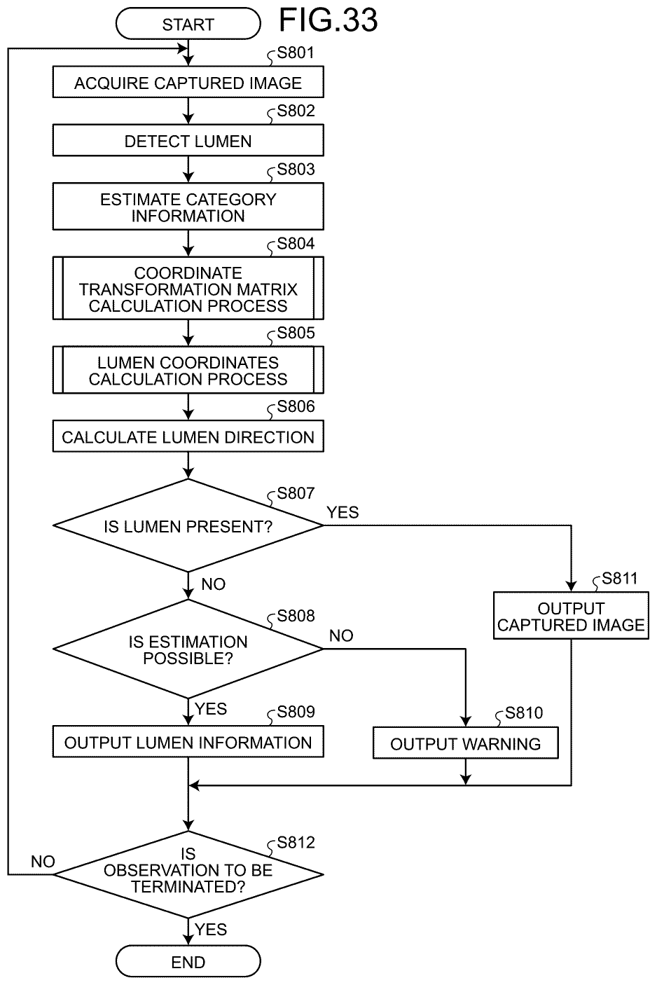

A process performed by the control device 4C will be described below. FIG. 33 is a flowchart illustrating an overview of a process that is performed by the control device 4C. In FIG. 33, Step S801, Step S803, Step S804, and Step S806 to Step S812 respectively correspond to Step S501 to Step S503 and Step S505 to Step S511 in FIG. 22 as described above, and Step S802 and Step S805 are different. In the following, Step S802 and Step S805 will be described.

At Step S802, the lumen detection unit 500 outputs presence or absence of a lumen in the captured image P1, from the captured image P1, by the CNN using the lumen detection trained model that is recorded in the lumen detection trained model recording unit 475, and outputs detected coordinates at which the lumen is detected when the lumen is present. After Step S802, the control device 4C goes to Step S803.

At Step S805, the lumen coordinates calculation unit 498 performs the lumen coordinates calculation process of calculating the lumen coordinates of the lumen in the captured image P1 based on presence or absence of a lumen and the detected coordinates that are input from the lumen detection unit 500, the category information that is input from the estimation unit 492, and the affine matrix and the trackability of the lumen tracking that are input from the coordinate transformation matrix calculation unit 497, outputting the lumen coordinates to the lumen direction calculation unit 499, and outputting the possibility of estimation of a lumen to the determination unit 493.

### Overview of Lumen Coordinates Calculation Process

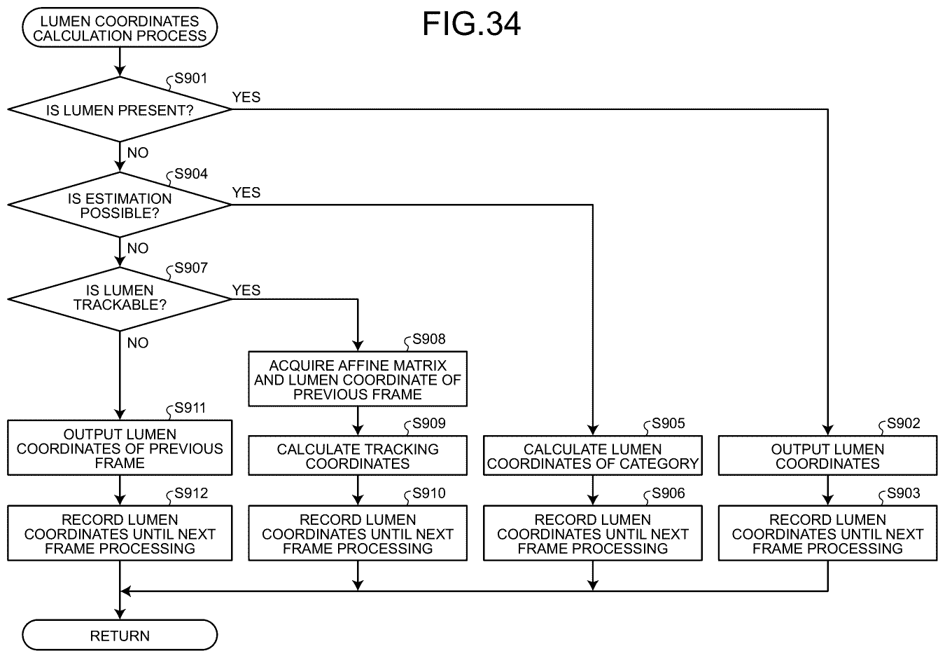

FIG. 34 is a flowchart illustrating an overview of the lumen coordinates calculation process. In FIG. 34, at Step S904 to Step S912, the same processes as the processes Step S701 to Step S709 in FIG. 24 as described above are performed, and Step S901 to Step S903 are different. In the following, Step S901 to Step S903 will be described.

At Step S901 illustrated in FIG. 34, the lumen coordinates calculation unit 498 determines whether or not a lumen is present in the captured image based on a detection result that is detected by the lumen detection unit 500. When the lumen coordinates calculation unit 498 determines that a lumen is present in the captured image (Step S901: Yes), the control device 4C goes to Step S902 to be described later. In contrast, when the lumen coordinates calculation unit 498 determines that a lumen is absent in the captured image (Step S901: No), the control device 4C goes to Step S904.

At Step S902, the lumen coordinates calculation unit 498 outputs lumen coordinates based on presence or absence of the lumen and the detected coordinates of the lumen that are detected by the lumen detection unit 500. The lumen coordinates calculation unit 498

records the lumen coordinates calculated at Step S902 until next frame processing on the captured image (Step S903). After Step S903, the control device 4C returns and goes to Step S806 in FIG. 33.

According to the fourth embodiment as described above, even when a lumen is absent in the captured image, it is possible to output the lumen direction, and when a lumen is present in the captured image it is possible to output a position of the lumen with high accuracy.

### Fifth Embodiment

A fifth embodiment will be described below. In the fifth embodiment, lumen information is output in accordance with proficiency of endoscope operation of an operator. In the following, a functional configuration of a control device according to the fifth embodiment will be first described, and thereafter, a process performed by the control device according to the fifth embodiment will be described.

### Functional Configuration of Control Device

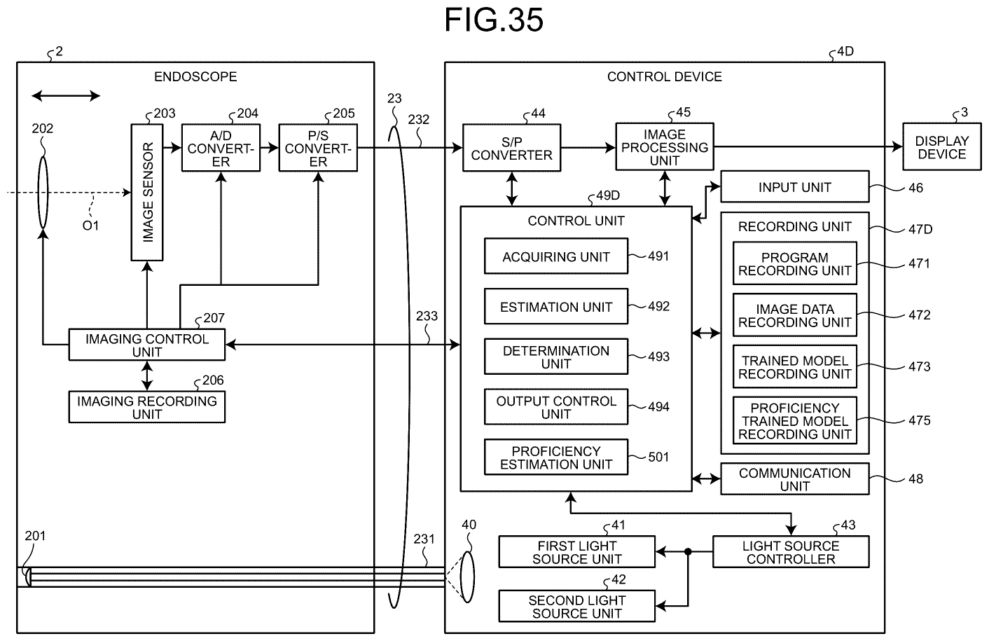

FIG. 35 is a block diagram illustrating a functional configuration of a main part of an endoscope and a control device according to the fifth embodiment. A control device 4D illustrated in FIG. 35 includes a recording unit 47D and a control unit 49D, instead of the recording unit 47 and the control unit 49 of the control device 4 according to the first embodiment as described above.

The recording unit 47D further includes a proficiency trained model recording unit 476, in addition to the functional configuration of the recording unit 47 according to the first embodiment as described above.

The proficiency trained model recording unit 476 records therein a proficiency trained model that has performed training on training data in which each of training images that correspond to pieces of captured data that are captured by the endoscope 2 is associated with lumen presence-absence information that differs depending on proficiency of an operator with respect to an endoscope. The proficiency trained model is generated by using the CNN that is described above in the first embodiment.

The control unit 49D further includes a proficiency estimation unit 501, in addition to the functional configuration of the control unit 49 according to the first embodiment as described above.

The proficiency estimation unit 501 inputs the captured image to the CNN using the proficiency trained model that is recorded in a proficiency trained model recording unit 476, and causes the proficiency of the operator and perform estimation to be output.

### Process Performed by Control Device

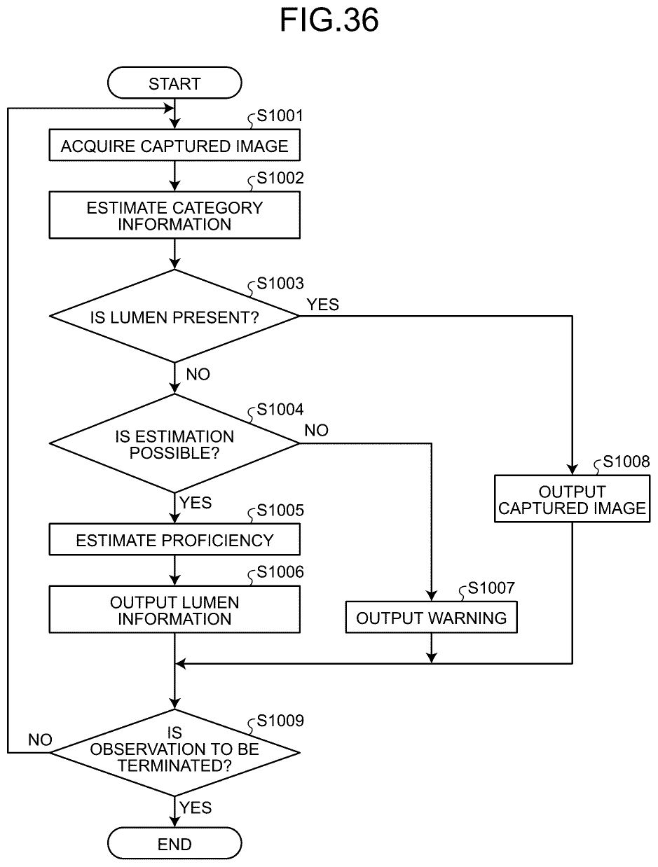

A process performed by the control device 4D will be described below. FIG. 36 is a flowchart illustrating an overview of a process that is performed by the control device 4D. In FIG. 36, Step S1001 to Step S1004 and Step S1007 to Step S1009 respectively correspond to Step S101 to Step S104 and Step S106 to Step S108, and Step S1005 and Step S1006 are different. In the following, Step S1005 and Step S1006 will be described.

At Step S1005, the proficiency estimation unit 501 inputs the captured image to the CNN using the proficiency trained model that is recorded in the proficiency trained model recording unit 476, causes the proficiency of the operator to be output, and performs estimation.

Subsequently, the output control unit 494 superimposes the lumen information that corresponds to the category information in accordance with the proficiency of the operator onto the captured image P1, and outputs the captured image with the lumen information to the display device 3 (Step S1006). After Step S1006, the control device 4D goes to Step S107.

According to the fifth embodiment as described above, the output control unit 494 superimposes the lumen information that corresponds to the category information in accordance with the proficiency of the operator onto the captured image P1, and outputs the captured image with the lumen information to the display device 3; therefore, it is possible to assist observation in accordance with the proficiency of the operator.

Meanwhile, in the fifth embodiment, the output control unit 494 superimposes the lumen information that corresponds to the category information in accordance with the proficiency of the operator onto the captured image P1, and outputs the captured image with the lumen information to the display device 3, but embodiments are not limited to this example. For example, it may be possible to use a common trained model, and when the determination unit 493 determines presence or absence of a lumen, it may be possible to adjust a threshold for a degree of reliability in accordance with the proficiency. For example, the determination unit 493 may adjust the threshold for the degree of reliability in accordance with an estimation result that is estimated by the proficiency estimation unit 501 by inputting the captured image to the CNN using the proficiency trained model that is recorded in the proficiency trained model recording unit 476, causing the proficiency of the operator to be output, and performing estimation. For example, the determination unit 493 may perform determination by setting a larger threshold such that a lumen is determined as being present with an increase in the proficiency.

### Other Embodiment

Various inventions may be made by appropriately combining a plurality of components disclosed in the endoscopes according to the first embodiment to the fifth embodiment of the present disclosure as described above. For example, some of the components may be removed from all of the components described in the endoscope system according to one embodiment of the present disclosure as described above. In addition, components described in the endoscope system according to embodiments of the present disclosure as described above may be combined appropriately.

Furthermore, in the endoscope system according to the first embodiment to the fifth embodiment of the present disclosure, the control devices 4 and 4A to 4D have functions to estimate a lumen direction and output the lumen direction, and include the trained model recording unit 473, the acquiring unit 491, the estimation unit 492, the determination unit 493, and the output control unit 494; however, for example, the trained model recording unit 473, the acquiring unit 491, the estimation unit 492, the determination unit 493, and the output control unit 494 may be arranged in a different support device or a different image processing device other than the control devices 4 and 4A to 4D. It is of course possible that the endoscope system according to the first embodiment to the fifth embodiment of the present disclosure may be implemented by a server that includes the trained model recording unit 473, the acquiring unit 491, the estimation unit 492, the determination unit 493, and the output control unit 494 via a network. It is of course possible that a server is assigned to each of the functions and each of the processes is performed in a distributed manner.

Moreover, in the endoscope system according to the first embodiment to the fifth embodiment of the present disclosure, the “unit” as described above may be replaced with a “means”, a “circuit”, or the like. For example, the control unit may be replaced with a control means or a control circuit.

Furthermore, a program that is executed by the endoscope system according to the first embodiment to the fifth embodiment of the present disclosure is provided by being recorded in a computer readable recording medium, such as a compact disc-read only memory (CD-ROM), a flexible disk (FD), a CD-recordable (CD-R), a Digital Versatile Disk (DVD), a universal serial bus (USB) medium, or a flash memory, as installable or executable file data.

Moreover, a program that is executed by the endoscope system according to the first embodiment to the fifth embodiment of the present disclosure may be stored in a computer connected to a network, such as the Internet, and may be provided by download via the network

Meanwhile, in the description of the flowcharts in the present specification, context of the processes among the steps is disclosed by using expressions such as “first”, “thereafter”, and “subsequently”, but the sequences of the processes needed to carry out the present disclosure are not uniquely defined by these expressions. In other words, the sequences of the processes in the flowcharts described in the present specification may be modified as long as there is no contradiction.

According to the present disclosure, it is possible to present a lumen direction even when a lumen is absent in an image.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

### Example

An image processing device comprising:
a processor configured to
   acquire a captured image corresponding to captured image data that is captured by an endoscope in a body cavity of a subject,
   input the captured image to a Convolutional Neural Network (CNN) using a trained model that has performed training on training data in which each of training images that are captured by the endoscope in the body cavity of the subject is associated with category information that includes at least presence or absence of a lumen and a lumen direction indicating a direction that is divided for each predetermined region with respect to a position of the lumen with reference to a center of each training image,
   estimate category information that includes presence or absence of the lumen and a lumen direction indicating a position of a lumen that is estimated to be present outside the captured image when the captured image is divided for each predetermined region, and
   output the estimated category information.

What is claimed is:

1. An image processing device comprising:
one or more processors comprising hardware and configured to:
input a captured image captured by an endoscope in a body cavity of a subject to a Convolutional Neural Network (CNN) using a trained model, the trained model having training data in which each of training images is associated with category information including a lumen direction of a region outside the each of training images in which the lumen is present;
estimate, based on the CNN, category information including a lumen direction of a region outside the captured image in which the lumen is likely to be present; and
output the category information estimated.

2. The image processing device according to claim 1, wherein the one or more processors is configured to:
estimate absence of the lumen in the captured image;
in response to estimating the category information including absence of the lumen in the captured image,
superimpose lumen information on the lumen direction of the region outside the captured image in which the lumen is likely to be present onto either the captured image or a peripheral region of the captured image; and
output either the captured image or the peripheral region of the captured image with the superimposed lumen information.

3. The image processing device according to claim 1, wherein:
the category information further includes lumen directions of regions outside the captured image in which the lumen is likely to be present and a degree of reliability of each of the lumen directions; and
the one or more processors are configured to:
superimpose lumen information onto a position of the captured image that corresponds to the lumen direction for which the degree of reliability is the highest; and
output the captured image with the superimposed lumen information.

4. The image processing device according to claim 1, wherein:
the category information further includes lumen directions of regions outside the captured image in which the lumen is likely to be present and a degree of reliability of each of the lumen directions; and
the one or more processors are configured to:
determine whether each of the degrees of reliability is equal to or larger than a threshold; and
in response to determining that the each of the degrees of reliability is not equal to or larger than the threshold, output a warning for instructing a change of an image capturing direction in which the endoscope captures an image.

5. The image processing apparatus according to claim 2, wherein the lumen information includes one or more of a figure, a character, and a frame that encloses the captured image.

6. The image processing device according to claim 1, wherein the one or more processors are configured to:
acquire a plurality of captured images that are sequentially captured by the endoscope;
estimate, based on the CNN, the category information of each of the plurality of captured images sequentially; and
for each of the plurality of captured images:
superimpose lumen information on the lumen direction of the region outside the each of the plurality of captured images in which the lumen is likely to be present onto the captured image while gradually decreasing at least one of a color and brightness with passage of time; and
output the each of the plurality of captured images with the superimposed lumen information.

7. The image processing device according to claim 1, wherein:
the category information further includes a degree of reliability of the lumen direction; and
the one or more processors are configured to:
superimpose the lumen information and the degree of reliability onto the captured image; and
output the captured image with the superimposed lumen information and the superimposed degree of reliability.

8. The image processing device according to claim 1, wherein:
the trained model is provided for each of regions in a subject; and
the one or more processors are further configured to:
input the captured image to a CNN using a region trained model having training data in which each of training images is associated with a captured image of a predetermined region;
estimate, based on the CNN using the region trained model, that the captured image was captured in the predetermined region.

9. The image processing device according to claim 1, wherein the one or more processors are configured to:
detect, as a plurality of corresponding points, corresponding pixel positions between chronologically successive captured images based on chronologically successive captured images;
identify coordinates of observation positions in the chronologically successive captured images based on the chronologically successive captured images,
convert a coordinate of an observation position identified in the captured image of a previous frame into a coordinate in a coordinate system of the captured image of a current frame based on the plurality of corresponding points;
determine whether the coordinates of the observation positions in chronologically successive captured images are trackable based on the corresponding points in the chronologically successive captured images;
determine whether it is possible to estimate the lumen direction based on the estimated category information, and
in response to determining the lumen direction cannot be estimated and it is possible to track the coordinates of the observation positions, covert the coordinate of the observation position of the previous frame into the coordinate in the coordinate system of the captured image of the current frame to estimate the lumen direction.

10. The image processing device according to claim 9, wherein the one or more processors are configured to:
input the captured image to a CNN using a lumen detection trained model having training data in which each of the training images is associated with presence or absence of a lumen and a coordinate of a position of a lumen in each of the training images;
estimate, based on the CNN, presence or absence of a lumen in the captured image and a lumen coordinate indicating a position of the lumen; and
in response to estimating the presence of the lumen in the captured image, output the lumen coordinate.

11. The image processing device according to claim 1, wherein the one or more processors are configured to:
input the captured image to a CNN using a trained model for each proficiency, the trained model having training data in which each of the training images is associated with lumen presence-absence information that differs depending on proficiency of an operator with respect to an endoscope; and
output the category information estimated in accordance with the proficiency of the operator.

12. The image processing device according to claim 1, wherein the one or more processors are configured to estimate absence of the lumen.

13. An image processing method implemented by one or more processors, the image processing method comprising:
inputting a captured image captured by an endoscope in a body cavity of a subject to a Convolutional Neural Network (CNN) using a trained model, the trained model having training data in which each of training images is associated with category information that includes a lumen direction of a region outside the each of training images in which the lumen is present;
estimating, based on the CNN, category information including a lumen direction of a region outside the captured image in which the lumen is likely to be present; and
outputting the category information estimated.

14. A non-transitory computer readable recording medium having recorded therein a program, the program when executed, causing a computer to perform:
inputting a captured image captured by an endoscope in a body cavity of a subject to a Convolutional Neural Network (CNN) using a trained model, the trained model having training data in which each of training images is associated with a category information that includes a lumen direction of a region outside the each of training images in which the lumen is present;
estimating, based on the CNN, category information including a lumen direction of a region outside the captured image in which the lumen is likely to be present; and
outputting the category information estimated.

15. The image processing method according to claim 13, further comprising:
estimate absence of the lumen.

16. The image processing method according to claim 13, further comprising:
estimate absence of the lumen in the captured image;
in response to estimating the category information including absence of the lumen in the captured image, superimpose lumen information on the lumen direction of the region outside the captured image in which the lumen is likely to be present onto either the captured image or a peripheral region of the captured image; and
output either the captured image or the peripheral region of the captured image with the superimposed lumen information.

17. The image processing method according to claim 13, wherein
the category information further includes lumen directions of regions outside the captured image in which the lumen is likely to be present and a degree of reliability of each of the lumen directions; and
the image processing method further comprising:
superimpose lumen information onto a position of the captured image that corresponds to the lumen direction for which the degree of reliability is the highest; and
output the captured image with the superimposed lumen information.

18. The image processing method according to claim 13, wherein
the category information further includes lumen directions of regions outside the captured image in which the lumen is likely to be present and a degree of reliability of each of the lumen directions; and
the image processing method further comprising:
determine whether each of the degrees of reliability is equal to or larger than a threshold; and
in response to determining that the each of the degrees of reliability is not equal to or larger than the threshold, output a warning for instructing a change of an image capturing direction in which the endoscope captures an image.

19. The image processing method according to claim 18, wherein the lumen information includes one or more of a figure, a character, and a frame that encloses the captured image.

20. The image processing method according to claim 13, further comprising:
acquire a plurality of captured images that are sequentially captured by the endoscope;
estimate, based on the CNN, the category information of each of the plurality of captured images sequentially; and
for each of the plurality of captured images:
superimpose lumen information on the lumen direction of the region outside the each of the plurality of captured images in which the lumen is likely to be present onto the captured image while gradually decreasing at least one of a color and brightness with passage of time; and
output the each of the plurality of captured images with the superimposed lumen information.
